(19) United States
(12) Patent Application Publication
(10) Pub. No.: US 2026/0108476 A1
(43) Pub. Date: Apr. 23, 2026

(54) 2,4 DNP and Prodrugs Thereof for Treatment of Cancer

(71) Applicants: Mitochon Pharmaceuticals, Inc., Blue Bell, PA (US); Northwestern University, Evanston, IL (US)

(72) Inventors: John Gerard GEISLER, Blue Bell, PA (US); Robert ALONSO, North Hampton, NH (US); Marcelo G. BONINI, Chicago, IL (US)

(73) Assignees: Mitochon Pharmaceuticals, Inc., Blue Bell, PA (US); Northwestern University, Evanston, IL (US)

(21) Appl. No.: 18/681,016

(22) PCT Filed: Aug. 4, 2022
(86) PCT No.: PCT/US2022/039419

§ 371 Date: Feb. 2, 2024

Related U.S. Application Data
(60) Provisional application No. 63/229,468, filed on Aug. 4, 2021.

(51) Int. Cl. A61K 31/06; A61K 31/203; A61K 31/255; A61K 31/337; A61K 31/4196; A61K 31/4453; A61K 31/495; A61K 31/519; A61K 31/53; A61K 31/5375; A61K 31/565; A61K 31/69; A61K 31/704; A61K 31/706; A61P 35/00

(52) U.S. Cl. CPC A61K 31/06; A61K 31/203; A61K 31/255; A61K 31/337; A61K 31/4196; A61K 31/4453; A61K 31/495; A61K 31/519; A61K 31/53; A61K 31/5375; A61K 31/565; A61K 31/69; A61K 31/704; A61K 31/706; A61P 35/00

(57) ABSTRACT

Compositions and methods of treatment of cancer including DNP and/or prodrugs of DNP are disclosed.

### CROSS REFERENCE TO PRIOR FILED APPLICATIONS

This application claims the benefit of priority to U.S. Provisional Patent Application No. 63/229,468, filed Aug. 4, 2021, which is incorporated by reference herein in its entirety.

### FIELD

The present disclosure relates to methods of treating cancer by administering DNP and/or prodrugs of DNP, or pharmaceutically acceptable salts thereof.

### BACKGROUND

Overt Reactive Oxygen Species (ROS) generated by mitochondria is a well-known byproduct of aging. With aging, mitochondria lose their ADP generating capacity and in turn release more ROSs that are known to be damaging to cells. This creates an environment that leads to cell death and degenerative processes.

Consequently, there is a profound need for improved treatments related to aging, such as cancer.

### SUMMARY

In one aspect, the disclosure provides a method of treating cancer. In some embodiments, the method includes administering to a patient in need thereof a therapeutic amount of a prodrug of dinitrophenol (DNP) or a pharmaceutically acceptable salt thereof, wherein the therapeutic amount of the prodrug of DNP is in the range of about 0.01 mg/kg of body weight to about 200 mg/kg of body weight.

In some embodiments, the therapeutic amount of DNP is in the range of 0.001 mg/kg of body weight to about 15 mg/kg of body weight, 0.01 mg/kg of body weight to about 15 mg/kg of body weight, 0.1 mg/kg of body weight to about 15 mg/kg of body weight, 0.001 mg/kg of body weight to about 0.1 mg/kg of body weight, 0.001 mg/kg of body weight to about 0.01 mg/kg of body weight, 0.01 mg/kg of body weight to about 0.1 mg/kg of body weight, about 1 mg/kg of body weight to about 10 mg/kg of body weight, about 3 mg/kg of body weight to about 8 mg/kg of body weight, or about 4 mg/kg of body weight to about 6 mg/kg of body weight.

In some embodiments, the therapeutic amount of DNP is about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, or about 10 mg/kg of body weight. In some embodiments, the therapeutic amount of DNP is about 0.001 mg/kg of body weight, about 0.002 mg/kg of body weight, about 0.003 mg/kg of body weight, about 0.004 mg/kg of body weight, about 0.005 mg/kg of body weight, about 0.006 mg/kg of body weight, about 0.007 mg/kg of body weight, about 0.008 mg/kg of body weight, about 0.009 mg/kg of body weight, or about 0.01 mg/kg of body weight.

In one aspect, the disclosure provides a method of treating cancer. In some embodiments, the method includes administering to a patient in need thereof a therapeutic amount of a prodrug of dinitrophenol (DNP) or a pharmaceutically acceptable salt thereof, wherein the therapeutic amount of the prodrug of DNP is in the range of about 0.01 mg/kg of body weight to about 200 mg/kg of body weight.

In some embodiments, the therapeutic amount of the prodrug of DNP is in the range of about 0.001 mg/kg of body weight to about 15 mg/kg of body weight, about 0.01 mg/kg of body weight to about 15 mg/kg of body weight, about 0.001 mg/kg of body weight to about 100 mg/kg of body weight, about 0.01 mg/kg of body weight to about 100 mg/kg of body weight, about 0.1 mg/kg of body weight to about 15 mg/kg of body weight, about 0.001 mg/kg of body weight to about 0.1 mg/kg of body weight, about 0.001 mg/kg of body weight to about 0.01 mg/kg of body weight, about 0.01 mg/kg of body weight to about 0.1 mg/kg of body weight, about 0.1 mg/kg of body weight to about 100 mg/kg of body weight, about 1 mg/kg of body weight to about 100 mg/kg of body weight, about 1 mg/kg of body weight to about 20 mg/kg of body weight, about 5 mg/kg of body weight to about 10 mg/kg of body weight, about 7 mg/kg of body weight to about 9 mg/kg of body weight, about 10 mg/kg of body weight to about 20 mg/kg of body weight, about 15 mg/kg of body weight to about 17 mg/kg of body weight, about 50 mg/kg of body weight to about 100 mg/kg of body weight, about 60 mg/kg of body weight to about 90 mg/kg of body weight, about 75 mg/kg of body weight to about 85 mg/kg of body weight, or about 79 mg/kg of body weight to about 81 mg/kg of body weight.

In some embodiments, the therapeutic amount of the prodrug of DNP is about 0.001 mg/kg of body weight, about 0.005 mg/kg of body weight, about 0.01 mg/kg of body weight, about 0.05 mg/kg of body weight, about 0.1 mg/kg of body weight, about 0.5 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, or about 20 mg/kg of body weight. In some embodiments, the therapeutic amount of the prodrug of DNP is about 0.001 mg/kg of body weight, about 0.002 mg/kg of body weight, about 0.003 mg/kg of body weight, about 0.004 mg/kg of body weight, about 0.005 mg/kg of body weight, about 0.006 mg/kg of body weight, about 0.007 mg/kg of body weight, about 0.008 mg/kg of body weight, about 0.009 mg/kg of body weight, about 0.01 mg/kg of body weight, about 0.011 mg/kg of body weight, about 0.012 mg/kg of body weight, about 0.013 mg/kg of body weight, about 0.014 mg/kg of body weight, about 0.015 mg/kg of body weight, about 0.016 mg/kg of body weight, about 0.017 mg/kg of body weight, about 0.018 mg/kg of body weight, about 0.019 mg/kg of body weight, or about 0.02 mg/kg of body weight.

In some embodiments, the therapeutic amount of the prodrug of DNP is about 71 mg/kg of body weight, about 72 mg/kg of body weight, about 73 mg/kg of body weight, about 74 mg/kg of body weight, about 75 mg/kg of body weight, about 76 mg/kg of body weight, about 77 mg/kg of body weight, about 78 mg/kg of body weight, about 79 mg/kg of body weight, 80 mg/kg of body weight, about 81 mg/kg of body weight, about 82 mg/kg of body weight, about 83 mg/kg of body weight, about 84 mg/kg of body weight, about 85 mg/kg of body weight, about 86 mg/kg of body weight, about 87 mg/kg of body weight, about 88 mg/kg of body weight, about 89 mg/kg of body weight, or about 90 mg/kg of body weight.

In some embodiments, the DNP is selected from 2,3-DNP, 2,4-DNP, 2,5-DNP, 2,6-DNP, 3,4-DNP, or 3,5-DNP. In some embodiments, the DNP is 2,4-DNP.

In some embodiments, the prodrug of DNP is selected from:
an amino acid (AA) ester of 2,3-DNP, an amino acid (AA) ester of 2,4-DNP, an amino acid (AA) ester of 2,5-DNP, an amino acid (AA) ester of 2,6-DNP, an amino acid (AA) ester of 3,4-DNP, or an amino acid (AA) ester of 3,5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10);
AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13);
amino carbamate 2,3-DNP prodrugs, amino carbamate 2,4-DNP prodrugs, amino carbamate 2,5-DNP prodrugs, amino carbamate 2,6-DNP prodrugs, amino carbamate 3,4-DNP prodrugs, or amino carbamate 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17);
amino carbonate 2,3-DNP prodrugs, amino carbonate 2,4-DNP prodrugs, amino carbonate 2,5-DNP prodrugs, amino carbonate 2,6-DNP prodrugs, amino carbonate 3,4-DNP prodrugs, or amino carbonate 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19);
phosphate analogs I-20, I-21, II-20, II-21, III-20, III-21, IV-20, IV-21, V-20, V-21, VI-20, and VI-21 (Schemes 1-6);
1,3 diketo analogs I-22 to I-32; II-22 to II-32; III-22 to III-32; IV-22 to IV-32; V-22 to V-32; and VI-22 to VI-32 (Schemes 1-6);
carbonate and carbamate analogs I-33 to I-39; II-33 to II-39; III-33 to III-39; IV-33 to IV-39; V-33 to V-39; and VI-33 to VI-39 (Schemes 1-6); and
benzoate analogs I-40, II-40, III-40, IV-40, V-40, and VI-40 (Schemes 1-6);
and combinations thereof.

In some embodiments, the prodrug of DNP is selected from:

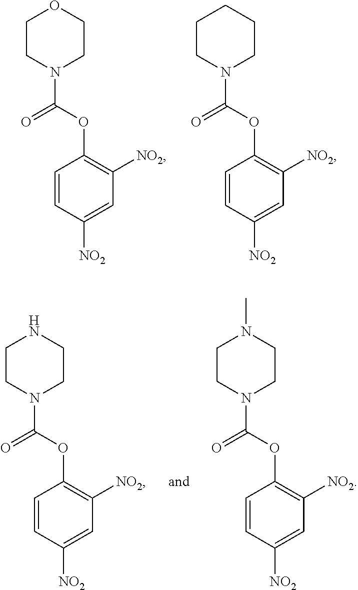

In some embodiments, the DNP and/or the prodrug of DNP is administered orally. some embodiments, the DNP and/or the prodrug of DNP is administered intravenously, subcutaneously, or transdermally.

In some embodiments, the DNP and/or the prodrug of DNP is administered at a frequency of once a day, twice a day, three times a day, four times a day, once every two days, once every three days, once every four days, once every five days, once every six days, once a week, once every two weeks, once every three weeks, or once a month. In some embodiments, the DNP and/or the prodrug of DNP is administered at a frequency of once a day. In some embodiments, the DNP and/or the prodrug of DNP is administered at a frequency of once a day for one day, once a day for two consecutive days, once a day for three consecutive days, once a day for four consecutive days, once a day for five consecutive days, once a day for six consecutive days, once a day for 7 consecutive days, or once a day for two consecutive weeks. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the DNP and/or the prodrug of DNP is administered in combination with an additional therapeutic agent. In some embodiments, the additional therapeutic agent comprises chemotherapy. In some embodiments, the additional therapeutic agent is selected from abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezomib, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dalteparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, paclitaxel, pegaspargase, pegfilgrastim, peginterferon alpha-2b, pemetrexed, pentostatin, pipobroman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the additional therapeutic agent is doxorubicin or an analogue thereof. In some embodiments, the additional therapeutic agent is fulvestrant or an analogue thereof. In some embodiments, the additional therapeutic agent is paclitaxel or an analogue thereof. In some embodiments, the additional therapeutic agent is docetaxel or an analogue thereof. In some embodiments, the DNP and/or the prodrug of DNP is administered without chemotherapy. In some embodiments, the additional therapeutic agent comprises radiation therapy.

### BRIEF DESCRIPTION OF THE DRAWINGS

The foregoing summary, as well as the following detailed description of embodiments of the disclosure, are better understood when read in conjunction with the appended drawings and figures.

FIGS. 1A-1D show elevated levels of nuclear ROS in aggressive cancer cells are linked to EMT and proliferation. FIG. 1A is a collection of images showing NLS-Orp1roGFP1 was used to quantify levels of nuclear ROS (shown as heat maps) in non-transformed (MCF10A) as well as tumorigenic (MCF7) and metastatic (BT474, BT20, MB231) breast cancer cell lines.

FIG. 1B is a graph of experimental data showing the quantification of the relative levels of nuclear ROS shown as the ratio of fluorescence between the oxidized and reduced forms. FIG. 1C is a Western blot analysis showing increased EMT markers after activation of nuclear ROS with D-Ala (10 nM). FIG. 1D is a graph of experimental data showing proliferation curves comparing naïve (MCF10A) and D-Ala (10 nM) exposed MCF10A/NLS-DAO cells. Results are averages of at least 5 biologic replicates with (*) representing p<0.05.

FIGS. 2A-2B show heightened levels of nuclear ROS in persister chemotherapy-resistance cancer cells. FIG. 2A is a collection of images and graphs showing experimental data generated using NLS-Orp1roGFP2 biosensor to measure ROS in the nucleus of breast cancer cell lines. PY230 murine mammary tumor cells expressing NLS-Orp1roGFP2 were treated with doxorubicin (DXR, 250 nM) for the indicated amounts of times in plots of proliferation as well as fluorescence ratios indicate that persister cells have high levels of nuclear ROS and proliferate in the presence of chemotherapy mimicking clinical chemotherapy resistance where most cells initially die leaving persister ones that rapidly proliferate unabated by treatment. Induction of NLS-catalase, that removes ROS from the nucleus, resensitized persister cells to the killing effects of DXR. FIG. 2B is a collection of images showing mammary xenografts of luciferase-labeled transformed MCF10A constitutively expressing NLS-catalase under an inducible promoter rapidly grow and metastasize within 3 months of implantation. Activation of NLS-catalase expression on month 3 induced the remission of metastatic lesions but not primary tumor. Combination therapy with DXR starting month 5 led to near complete remission of tumorigenic lesions (primary and metastatic).

FIGS. 3A-3C show how DNP suppresses nuclear ROS in basal BT breast cancer cell lines. FIGS. 3A-3B are graphs showing the quantification of levels of ROS in each cell nuclei showing a clear concentration dependence of nROS reduction. FIG. 3A shows BT20 NLS Orp roGFP cells and FIG. 3B shows BT474 NLS Orp roGFP cells, both with varying concentrations of DNP for 24 hours. FIG. 3C shows representative fluorescence micrographs showing ox/red Orp1roGFP2 ratios before and after treatment with MP101 (100 μM, 24 h), *p<0.05; **p<0.01; ***p<0.001 and ***p<0.0001.

FIG. 4 show images of PWS nanoimage of live Hela cells. Pseudocolor: heterogeneity of macro-molecular density with sensitivity to length scales from 20-200 nm. N=nuclei.

FIGS. 5A-5D show that variations in chromatin folding modulate transcriptional heterogeneity. FIG. 5A is a graph showing structural alterations due to taxol treatment (Paclitaxel or Docetaxel) in contrast to digoxin for five cell line models (A2780, M248, MES-SA, MX2). Chemotherapeutic intervention increases while CPT agent (digoxin) decreases chromatin folding. Error bars: S.E. of 5 different cell lines. FIGS. 5B-5C are diagrams showing intercellular (FIG. 5B) and intra-network (FIG. 5C) transcriptional heterogeneity increases in cells treated with chemotherapy and decreases in cells treated with CPTs for critical biological processes: (1) cell cycle, (2) apoptosis, (3) proliferation, (4) transcription, (5) signaling, (6) differentiation, (7) glycolysis, (8) translation, (9) ion transport, (10) metabolism, (11) oxidation/reduction, (12) stress response, and (13) nucleosome assembly. Circle size: the number of genes within a network/process. Color: % change in transcription heterogeneity compared to controls. FIG. 5D shows images representing live cell PWS. Digoxin reduces chromatin heterogeneity. Arrow: nuclei.

### DETAILED DESCRIPTION

### Definitions

Hereinafter, unless defined otherwise, the term “prodrug” refers to an inactive or partially active drug that is metabolically changed in the body to an active drug.

Hereinafter, the term “depot nanoparticle formulation” unless defined otherwise refers to a biodeliverable nanoparticle, comprising a broad range of 1) bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs, 2) Gemini prodrugs and 3) bioprecursor molecules for treating neurodegenerative or metabolic diseases.

Hereinafter, unless otherwise defined, the term “about” means plus or minus 10% of the value referenced. For example, “about 1 mg/kg” means 0.9 mg/kg to 1.1 mg/kg.

Hereinafter, unless defined otherwise, bipartite 2,3-dinitrophenol, 2,4-dinitrophenol, 2,5-dinitrophenol, 2,6-dinitrophenol, 3,4-dinitrophenol, or 3,5-dinitrophenol are represented by 2,3-DNP, 2,4-DNP, 2,5-DNP, 2,6-DNP, 3,4-DNP, or 3,5-DNP; the prodrug of each isomer is represented in formulas I-VI; being selected from the group consisting of:
an amino acid (AA) ester of 2,3-DNP, 2,4-DNP, 2,5-DNP, 2,6-DNP, 3,4-DNP, or 3,5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10);
AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13);
amino carbamate 2,3-DNP, 2,4-DNP, 2,5-DNP, 2,6-DNP, 3,4-DNP, or 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17);
amino carbonate 2,3-DNP, 2,4-DNP, 2,5-DNP, 2,6-DNP, 3,4-DNP, or 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19);
phosphate analogs I-20, I-21, II-20, II-21, III-20, III-21, IV-20, IV-21, V-20, V-21, VI-20, and VI-21 (Schemes 1-6);
1,3 diketo analogs I-22 to I-32; II-22 to II-32; III-22 to III-32; IV-22 to IV-32; V-22 to V-32; and VI-22 to VI-32 (Schemes 1-6);
carbonate and carbamate analogs I-33 to I-39; II-33 to II-39; III-33 to III-39; IV-33 to IV-39; V-33 to V-39; and VI-33 to VI-39 (Schemes 1-6);
benzoate analogs I-40, II-40, III-40, IV-40, V-40, and VI-40 (Schemes 1-6);
and combinations thereof.

In one aspect, the disclosure provides compositions and methods for treating cancer. In some embodiments, the compositions and methods include dinitrophenol (DNP) and/or prodrugs of DNP.

The production of Reactive Oxygen Species (ROS) may also drive new mutations in aging populations and thus increase the incidence of highly resistant cancers and pain-especially as they relate to aging.

Metastasis is the spread of cancer cells from the place where the tumor first appeared (primary tumor) to other sites in the body. While primary tumors are often curable, metastasis is difficult to manage and lethal. It has been found that mitochondrial ROS reaching the nucleus produces oxidative deformations to chromatin, causing structural alterations at the local level. The accumulation of such deformations affect the expression of multiple genes across the genome, including those directly associated with the activation of epithelial to mesenchymal transition (EMT), a process that generates highly plastic cancer cells. The stochastic nature of chromatin oxidation allows for significant heterogeneity in the response of tumor cells to progressive mitochondrial dysfunction, and leads to the diversification of transcriptomic states within cellular populations. Accordingly, it is hypothesized that suppressing mitochondrial ROS directly should be a major component of treatments focused on the management of aggressive cancer types to prevent the development of treatment resistance and minimize or reverse metastasis.

Cancer therapeutics have been revolutionized by the discovery of inhibitors targeting specific oncogenes or mutations driving tumor origination and progression. However, according to the American Cancer Society (ACS) only 5-10% of breast cancer cases are driven by genetic mutations while the vast majority occurs in postmenopausal women where metabolic dysfunction seems to be a contributing factor. This led to metabolic changes that happen with aging as a major driver of chromatin changes that alter normal patterns of gene expression to be the focus. The basic premise supporting this idea was founded on multiple studies of several groups that have shown that: (1) there is a decline in the mitochondrial metabolism with age parallel with an increase in ROS; (2) mitochondrial ROS production is significantly higher in cancer cells than in normal cells; (3) ROS reaching the nucleus produce alterations and mutations that promote aberrant gene expression leading to cancer initiation, progression and resistance to therapy. In addition, most recent studies indicated that nuclear ROS are both sufficient and required to trigger gene transcription associated with epithelial-to-mesenchymal transition (EMT), a process that initiates metastatic dissemination, colonization and often resistance to treatment. Hence, studies indicate that there is an underappreciated function for nuclear ROS in cancer that can be pharmacologically targeted to suppress treatment resistance and metastasis.

In aspects, the disclosure relates to the discovery that H₂O₂, a major form of ROS produced in mitochondria, reaches the nucleus and promotes oxidative chromatin remodeling that is both sufficient and required for EMT. While several cancer therapies aim at producing acute bursts of high level ROS to damage cancer cells, data indicates, in some embodiments, that heightened chronic nuclear ROS in persister cancer cells endow them with both the resilience they need to survive therapy-induced bursts and the plasticity they require to establish metastatic disease. This finding is consistent with the well-established concept of preconditioning whereby intermittent or chronic low level insult prepares cells or organisms to withstand acute to high-intensity challenge.

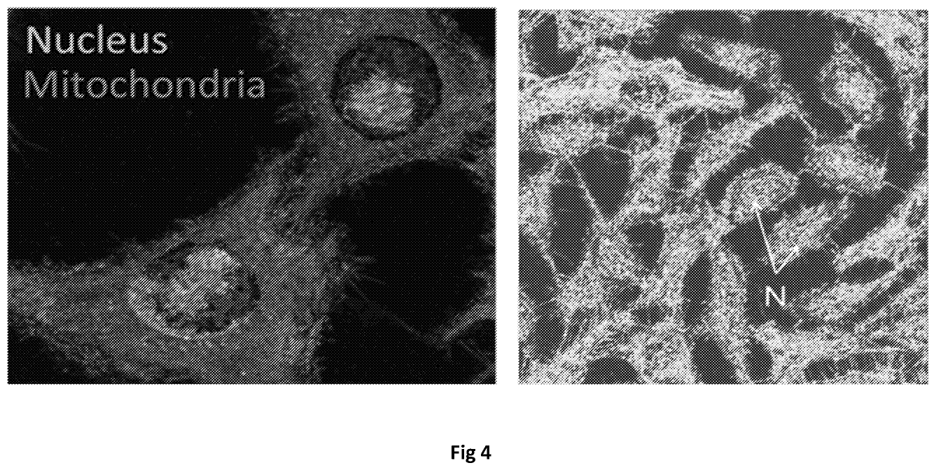

Chromatin is a complex arrangement of DNA wrapped around structural and regulatory proteins that control the expression of genes directly involved in cellular function and phenotype. Local physicochemical changes broadly affect chromatin folding and alter gene expression patterns across the genome. In a non-limiting example, decoding the chromatin folding code and understanding how different agents affect folding dynamics at the nanoscale can be helpful to develop new diagnostic tools. The resolution of optical microscopy is limited by diffraction to >200 nm. Recently, a major breakthrough has been made in the emergence of super-resolution fluorescence microscopy that enabled development of a suite of label-free nanoscale imaging technologies including Partial Wave Spectroscopic (PWS) Microscopy (see, for example, FIG. 4). The main principle of PWS is a previously overlooked phenomenon that while sub-diffractional structures are not resolvable, they are still detectable through the analysis of interference of elastically scattered light. PWS can measure the statistics of macromolecular density distribution within live or fixed cells with sensitivity in the 20-200 nm length scales, which is ideally suited for the characterization of chromatin topology. It was by using PWS (and corroborated by electron microscopy) that heterogenization of chromatin folding was discovered as one of the earliest events in carcinogenesis across multiple cancer types. Consequently, PWS was turned into a robust diagnostics technology (nanocytology) and is now in clinical trials for early screening or prognostication of lung, colon, and prostate cancers.

PWS studies validated by transmission electron microscopy (TEM) have demonstrated that an increased heterogeneity (Σ) and the fractal dimension (D) (Σ∝(D−D₀)δn)^{Δ} of chromatin folding is one of the earliest and most ubiquitous events in carcinogenesis across multiple types of cancer. Furthermore, D (fractal dimension) and Σ (heterogeneity) are increased in more aggressive tumors and are inversely correlated with survival time in patients with stage III-IV cancer.

In cancer cells it has been shown that mutations, especially in the elderly, are associated with a decline in mitochondrial function and an increase in Reactive Oxygen Species (ROS). Furthermore, it has been found that the increase in ROSs that reach the nucleus often leads to tumor cell heterogeneity and chemoresistance. This is also true with pain.

In one aspect, the disclosure provides the use of mitochondrial uncouplers, including 2,4 DNP and prodrugs thereof, as excellent therapies for the treatment of cancer and pain.

As described herein in a non-limiting example, treatment with DNP and/or prodrugs of DNP can abolish F2-isoprostanes, a biomarker of free radicals (reactive oxygen species) at the peak of disease disability (paralysis).

In one aspect, in cancer it has been found that DNP, such as 2,4 DNP, and prodrugs thereof prevent the accumulation of ROSs in the nucleus of cancer cells, and thus prevent cells from diversifying into a heterogenous phenotype as described above. Preventing heterogeneity through ROS prevention pushed cancer cells to remain homogenous and thus it was found that they are now more susceptible to traditional chemotherapies. In some embodiments, the treatment of cancer cell lines with DNP, such as 2,4 DNP, and prodrugs thereof prior to and during chemotherapy has been shown to create a more homogeneous environment as compared to standard therapies, and chemoresistance is prevented. Furthermore, mouse models of human cancers including, but not limited to, breast cancer (including pre and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer, have responded favorably to treatment with DNP, such as 2,4 DNP, and prodrugs thereof at minimizing heterogeneity and improving outcomes with and without standard chemotherapies.

In one aspect, the disclosure provides the use of DNP, such as 2,4 DNP, and prodrugs thereof in the treatment of cancer with and/or without standard chemotherapy.

In one aspect, the disclosure provides compositions and methods for treating pain and/or pain related conditions. In some embodiments, the compositions and methods include dinitrophenol (DNP) and/or prodrugs of DNP.

Diseases associated with pain, either acute, chronic, neuropathic, nociceptive, radicular pain or associated with syndromes (such as fibromyalgia) have a mitochondrial component of free radicals and inflammation. Chronic treatment using DNP, such as 2,4 DNP, and the prodrugs thereof, have attenuated progression or reversed pain where other drugs have failed. This platform for the treatment of pain is not associated with loss of cognition as found with opioids or other narcotics/psychotropics that cause confusion, clouded thinking, depression, drowsiness, sedation, etc. In fact, the treatment is associated with increased cognition through the induction of Brain Derived Neurotropic Factor (BDNF).

In one embodiment, the disclosure provides compositions and methods for treating diseases and disorders associated with high isoprostanes by administering DNP and/or prodrugs of DNP. Non-limiting examples of diseases and disorders associated with high isoprostanes include the cancers and/or the pain and pain related conditions described herein.

In one embodiment, the compositions described herein may be used to treat cancer. In one embodiment, the compositions described herein may be used to treat pain and/or a pain related condition. In one such embodiment, 2,4-dinitrophenol (DNP) may be used at a dose range independently selected from: 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day; about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; about 1 mg/day to about 50 mg/day; about 1 mg/day to about 45 mg/day; about 1 mg/day to about 40 mg/day; about 1 mg/day to about 35 mg/day; about 1 mg/day to about 30 mg/day; about 1 mg/day to about 25 mg/day; about 1 mg/day to about 20 mg/day; about 1 mg/day to about 15 mg/day; about 1 mg/day to about 10 mg/day; about 1 mg/day to about 5 mg/day; about 1 mg/day to about 4 mg/day; about 1 mg/day to about 3 mg/day; about 1 mg/day to about 2 mg/day; about 2 mg/day to about 50 mg/day; about 2 mg/day to about 45 mg/day; about 2 mg/day to about 40 mg/day; about 2 mg/day to about 35 mg/day; about 2 mg/day to about 30 mg/day; about 2 mg/day to about 25 mg/day; about 2 mg/day to about 20 mg/day; about 2 mg/day to about 15 mg/day; about 2 mg/day to about 10 mg/day; about 2 mg/day to about 5 mg/day; about 2 mg/day to about 4 mg/day; about 2 mg/day to about 3 mg/day; about 0.5 mg/day to about 10 mg/day; about 0.5 mg/day to about 9 mg/day; about 0.5 mg/day to about 8 mg/day; about 0.5 mg/day to about 7 mg/day; about 0.5 mg/day to about 6 mg/day; about 0.5 mg/day to about 5 mg/day; about 0.5 mg/day to about 4 mg/day; about 0.5 mg/day to about 3 mg/day; about 0.1 mg/day to about 10 mg/day; about 0.1 mg/day to about 9 mg/day; about 0.1 mg/day to about 8 mg/day; about 0.1 mg/day to about 7 mg/day; about 0.1 mg/day to about 6 mg/day; about 0.1 mg/day to about 5 mg/day; about 0.1 mg/day to about 4 mg/day; about 0.1 mg/day to about 3 mg/day. In one such embodiment, 2,4-dinitrophenol (DNP) may be used at a dose range independently selected from: about 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day; about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; 1 mg/day to about 50 mg/day; about 1 mg/day to about 45 mg/day; about 1 mg/day to about 40 mg/day; about 1 mg/day to about 35 mg/day; about 1 mg/day to about 30 mg/day; about 1 mg/day to about 25 mg/day; about 1 mg/day to about 20 mg/day; about 1 mg/day to about 15 mg/day; about 1 mg/day to about 10 mg/day; about 1 mg/day to about 5 mg/day; about 1 mg/day to about 4 mg/day; about 1 mg/day to about 3 mg/day; about 1 mg/day to about 2 mg/day; about 2 mg/day to about 50 mg/day; about 2 mg/day to about 45 mg/day; about 2 mg/day to about 40 mg/day; about 2 mg/day to about 35 mg/day; about 2 mg/day to about 30 mg/day; about 2 mg/day to about 25 mg/day; about 2 mg/day to about 20 mg/day; about 2 mg/day to about 15 mg/day; about 2 mg/day to about 10 mg/day; about 2 mg/day to about 5 mg/day; about 2 mg/day to about 4 mg/day; about 2 mg/day to about 3 mg/day; about 0.5 mg/day to about 10 mg/day; about 0.5 mg/day to about 9 mg/day; about 0.5 mg/day to about 8 mg/day; about 0.5 mg/day to about 7 mg/day; about 0.5 mg/day to about 6 mg/day; about 0.5 mg/day to about 5 mg/day; about 0.5 mg/day to about 4 mg/day; about 0.5 mg/day to about 3 mg/day; about 0.1 mg/day to about 10 mg/day; about 0.1 mg/day to about 9 mg/day; about 0.1 mg/day to about 8 mg/day; about 0.1 mg/day to about 7 mg/day; about 0.1 mg/day to about 6 mg/day; about 0.1 mg/day to about 5 mg/day; about 0.1 mg/day to about 4 mg/day; about 0.1 mg/day to about 3 mg/day; or about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.1 mg/day, about 1.2 mg/day, about 1.3 mg/day, about 1.4 mg/day, about 1.5 mg/day, about 1.6 mg/day, about 1.7 mg/day, about 1.8 mg/day, about 1.9 mg/day, about 2 mg/day, about 2.1 mg/day, about 2.2 mg/day, about 2.3 mg/day, about 2.4 mg/day, about 2.5 mg/day, about 2.6 mg/day, about 2.7 mg/day, about 2.8 mg/day, about 2.9 mg/day, about 3 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day, about 8 mg/day, about 8.1 mg/day, about 8.2 mg/day, about 8.3 mg/day, about 8.4 mg/day, about 8.5 mg/day, about 8.6 mg/day, about 8.7 mg/day, about 8.8 mg/day, about 8.9 mg/day, about 9 mg/day, about 9.1 mg/day, about 9.2 mg/day, about 9.3 mg/day, about 9.4 mg/day, about 9.5 mg/day, about 9.6 mg/day, about 9.7 mg/day, about 9.8 mg/day, about 9.9 mg/day, or about 10 mg/day.

In another such embodiment to treat cancer, a prodrug of 2,3-DNP, 2,4-DNP, 2,5-DNP, 2,6-DNP, 3,4-DNP, or 3,5-DNP, as described herein, may be used at a dose range to achieve equivalent exposure (AUC) to DNP. In another such embodiment to treat pain and/or a pain related condition, a prodrug of 2,3-DNP, 2,4-DNP, 2,5-DNP, 2,6-DNP, 3,4-DNP, or 3,5-DNP, as described herein, may be used at a dose range to achieve equivalent exposure (AUC) to DNP. In one such embodiment, a prodrug of 2,3-DNP, 2,4-DNP, 2,5-DNP, 2,6-DNP, 3,4-DNP, or 3,5-DNP, as described herein, may be used at a dose range independently selected from: 20 mg/day to about 800 mg/day; about 20 mg/day to about 750 mg/day; about 20 mg/day to about 700 mg/day; about 20 mg/day to about 600 mg/day; about 20 mg/day to about 700 mg/day; about 20 mg/day to about 600 mg/day; about 20 mg/day to about 500 mg/day; about 30 mg/day to about 800 mg/day; about 30 mg/day to about 700 mg/day; about 30 mg/day to about 600 mg/day; about 30 mg/day to about 500 mg/day; about 30 mg/day to about 400 mg/day; about 30 mg/day to about 360 mg/day; about 30 mg/day to about 300 mg/day; about 30 mg/day to about 250 mg/day; about 30 mg/day to about 200 mg/day; about 30 mg/day to about 150 mg/day; about 30 mg/day to about 100 mg/day; about 35 mg/day to about 360 mg/day; about 40 mg/day to about 300 mg/day; about 50 mg/day to about 250 mg/day; about or 60 mg/day to about 200 mg/day; about 5 mg/day to about 500 mg/day; about 5 mg/day to about 400 mg/day; about 5 mg/day to about 300 mg/day; about 5 mg/day to about 200 mg/day; about 5 mg/day to about 100 mg/day; about 5 mg/day to about 50 mg/day; about 5 mg/day to about 40 mg/day; or about 5 mg/day to about 30 mg/day. In another such embodiment, a prodrug of 2,3-DNP, 2,4-DNP, 2,5-DNP, 2,6-DNP, 3,4-DNP, or 3,5-DNP, as described herein, may be used at a dose range independently selected from: about 20 mg/day to about 800 mg/day; about 20 mg/day to about 750 mg/day; about 20 mg/day to about 700 mg/day; about 20 mg/day to about 600 mg/day; about 20 mg/day to about 700 mg/day; about 20 mg/day to about 600 mg/day; about 20 mg/day to about 500 mg/day; about 30 mg/day to about 800 mg/day; about 30 mg/day to about 700 mg/day; about 30 mg/day to about 600 mg/day; about 30 mg/day to about 500 mg/day; about 30 mg/day to about 400 mg/day; about 30 mg/day to about 360 mg/day; about 30 mg/day to about 300 mg/day; about 30 mg/day to about 250 mg/day; about 30 mg/day to about 200 mg/day; about 30 mg/day to about 150 mg/day; about 30 mg/day to about 100 mg/day; about 35 mg/day to about 360 mg/day; about 40 mg/day to about 300 mg/day; about 50 mg/day to about 250 mg/day; or about 60 mg/day to about 200 mg/day; about 5 mg/day to about 500 mg/day; about 5 mg/day to about 400 mg/day; about 5 mg/day to about 300 mg/day; about 5 mg/day to about 200 mg/day: about 5 mg/day to about 100 mg/day; about 5 mg/day to about 50 mg/day; about 5 mg/day to about 40 mg/day; or about 5 mg/day to about 30 mg/day.

In one embodiment, the compositions and methods of the disclosure relate to targeting oxidative stress upstream at the mitochondria that may be causative for many disorders, including, but not limited to, cancer, pain, and/or pain related conditions. There have been a variety of attempts to lower cellular stress, such as administering anti-oxidants, however these drugs have limited tissue penetration into the brain and lower ROSs after they have been formed. It has been observed that wildtype mice chronically treated with an extraordinarily low dose of 2, 4-dinitrophenol resulted in treated mice living longer than untreated mice. While not being bound to theory, it is postulated that DNP modulates the mitochondrial membrane potential having a significant impact at preventing ROS formation in isolated mitochondria and in the treated wildtype mice. DNP treatment 3-hours post-ischemia reduced cerebral infarct volume 40% by protecting the penumbra or “threaten tissue” and could provide similar benefits after a blast to protect inner hair cells from eminent cell death. DNP comes with the benefits of known risks as it was used 80-years ago for weight loss at high doses (˜300 mg) in over 100,000 people, however shown recently in models of neurodegeneration at very low hermetic doses to improve cognition and learning. The pharmacology, which is pleiotropic, appears to provide broad neuroprotection by lowering ROS/mTOR and increasing protective factors such as CAMP, CREB, and BDNF, could be useful for treating hearing loss. Despite DNP tainted past and common incorrect dogma of being toxic, a 28-day toxicity studies was ran and demonstrated that low doses of DNP are not toxic, have at least a 10× Safety Index with no inhibition to ion channels, CYP, Caco-2, etc., and provide therapeutically striking benefits in host CNS models representing diverse indications, thereby suggesting possible merit for inner ear and central brain hearing mitochondrial dysfunction.

### Chemical Synthesis:

DNP prodrugs have been previously described. See, for example, WO 2017/155940, which is incorporated by reference herein in its entirety.

In one embodiment, prodrugs which contain a self-cleavable spacer and a water-solubilizing moiety are synthesized, to maintain the prodrug in a soluble form in the GI tract fluids, and which will then gradually revert to the parent drug without precipitation. In some embodiments, these compounds are 1,3 diketo analogs I-22 to I-32; II-22 to II-32; III-22 to III-32; IV-22 to IV-32; V-22 to V-32; and VI-22 to VI-32 (Schemes 1-6). The increased solubility of the prodrug and the high membrane permeability of the well-dispersed parent drug will provide a higher driving force for it to be readily absorbed via the intestinal lumen. Conversion of prodrug-to-parent drug involves a chemical cleavage at the self-cleavable spacer through a unique intramolecular cyclization-elimination reaction via imide formation under physiological conditions. The conversion time is tunable by modifying the structure of the solubilizing moiety, the bond length of the spacer, the pKa of the amine group, and the pH of the medium. Although the in silico predicted bioavailability may below, it is likely to be much higher when one takes into account the unique pH-dependent and tunable hydrolysis mechanism. Also, the generation of parent drug does not rely on enzyme action, which may be an advantage in dealing with genetic variability associated with enzymatic prodrug hydrolysis in plasma.

In other embodiments, one or more isomers of dinitrophenol, i.e., 2,3-DNP, 2,4-DNP, 2,5-DNP, 2,6-DNP, 3,4-DNP, or 3,5-DNP, and/or a broad range of 1) bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs, 2) Gemini prodrugs and 3) bioprecursor molecules, are used for treating neurodegenerative or metabolic diseases.

Synthesis of bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs may be performed; the prodrug being selected from the group consisting of:
an amino acid (AA) ester of 2,3-DNP, 2,4-DNP, 2,5-DNP, 2,6-DNP, 3,4-DNP, or 3,5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10);
AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13);
amino carbamate 2,3-DNP, 2,4-DNP, 2,5-DNP, 2,6-DNP, 3,4-DNP, or 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17);
amino carbonate 2,3-DNP, 2,4-DNP, 2,5-DNP, 2,6-DNP, 3,4-DNP, or 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19);
phosphate analogs I-20, I-21, II-20, II-21, III-20, III-21, IV-20, IV-21, V-20, V-21, VI-20, and VI-21 (Schemes 1-6);
1,3 diketo analogs I-22 to I-32; II-22 to II-32; III-22 to III-32; IV-22 to IV-32; V-22 to V-32; and VI-22 to VI-32 (Schemes 1-6);
carbonate and carbamate analogs I-33 to I-39; II-33 to II-39; III-33 to III-39; IV-33 to IV-39; V-33 to V-39; and VI-33 to VI-39 (Schemes 1-6);
benzoate analogs I-40, II-40, III-40, IV-40, V-40, and VI-40 (Schemes 1-6); and combinations thereof, wherein the prodrug is represented by formulas I-VI:

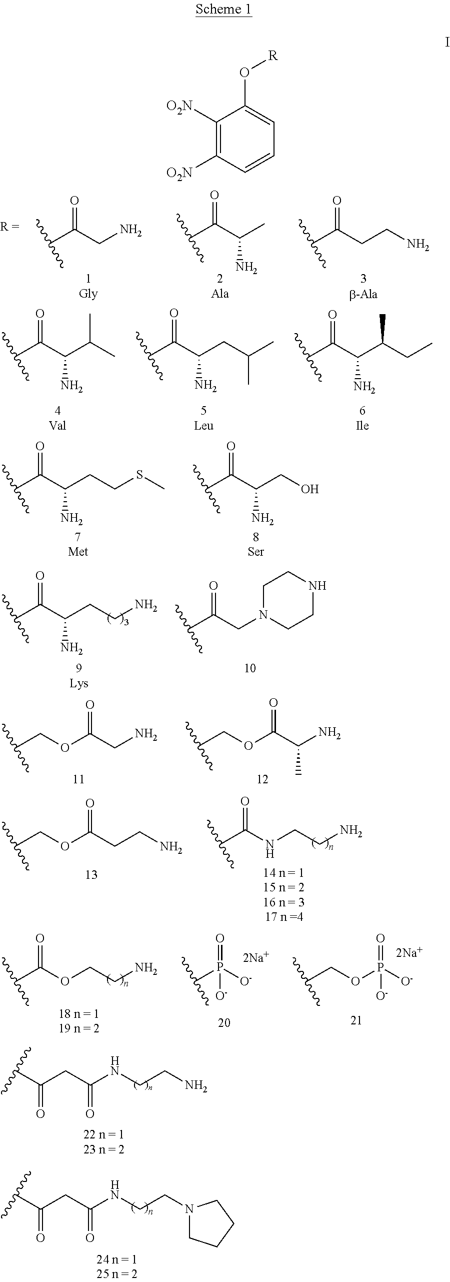

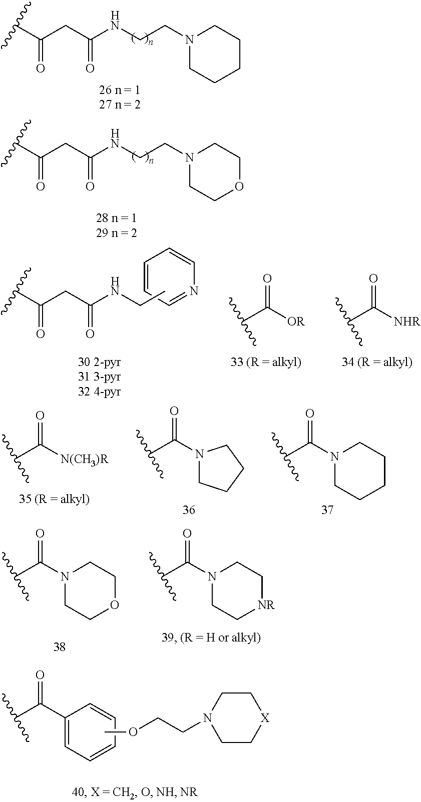

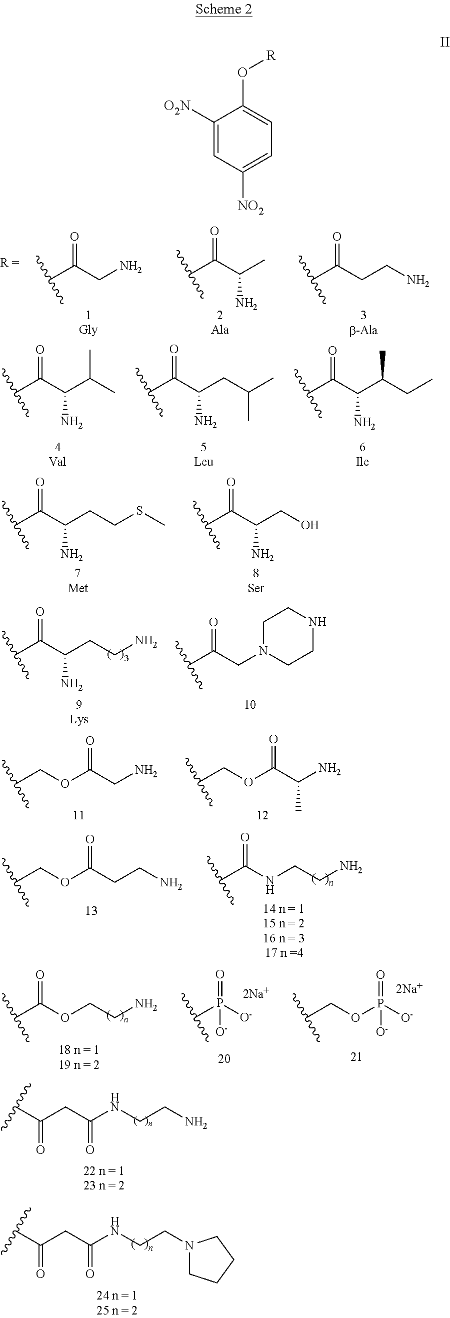

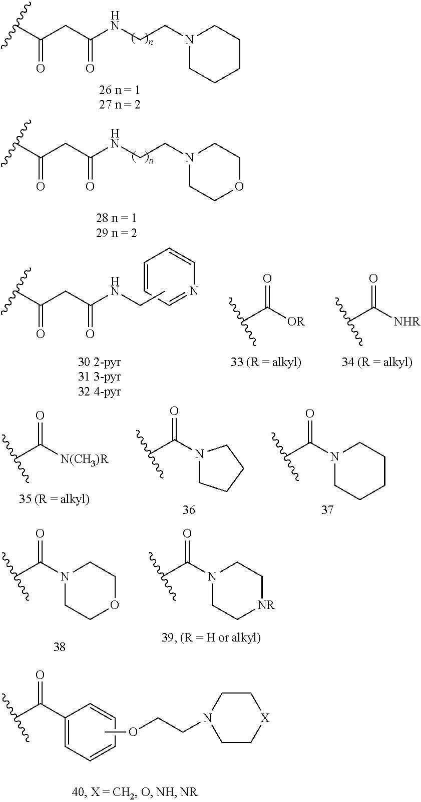

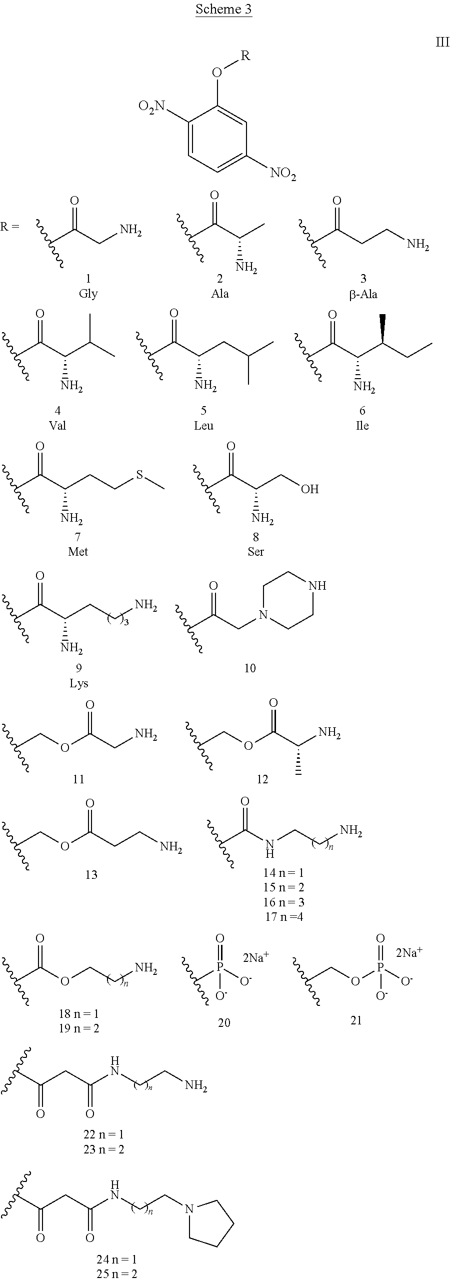

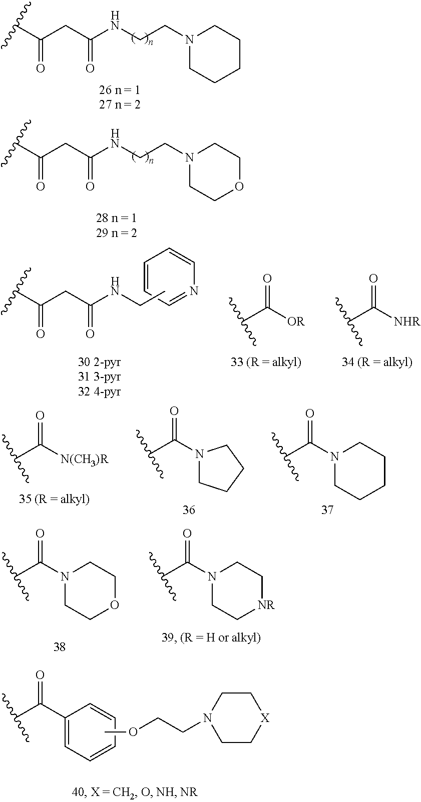

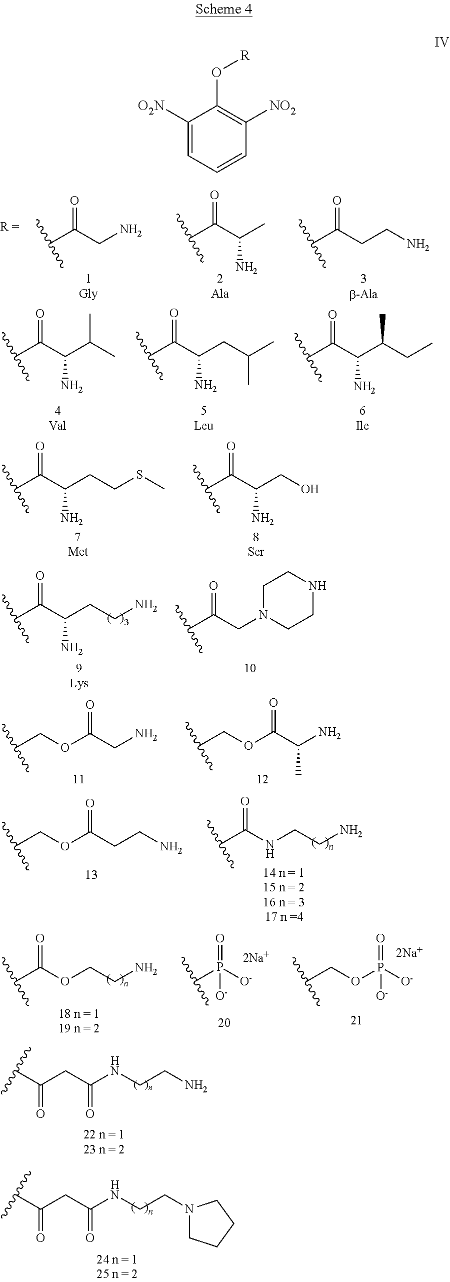

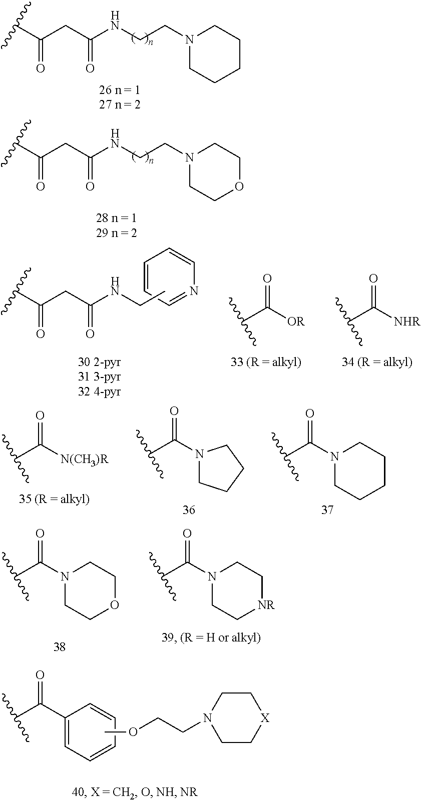

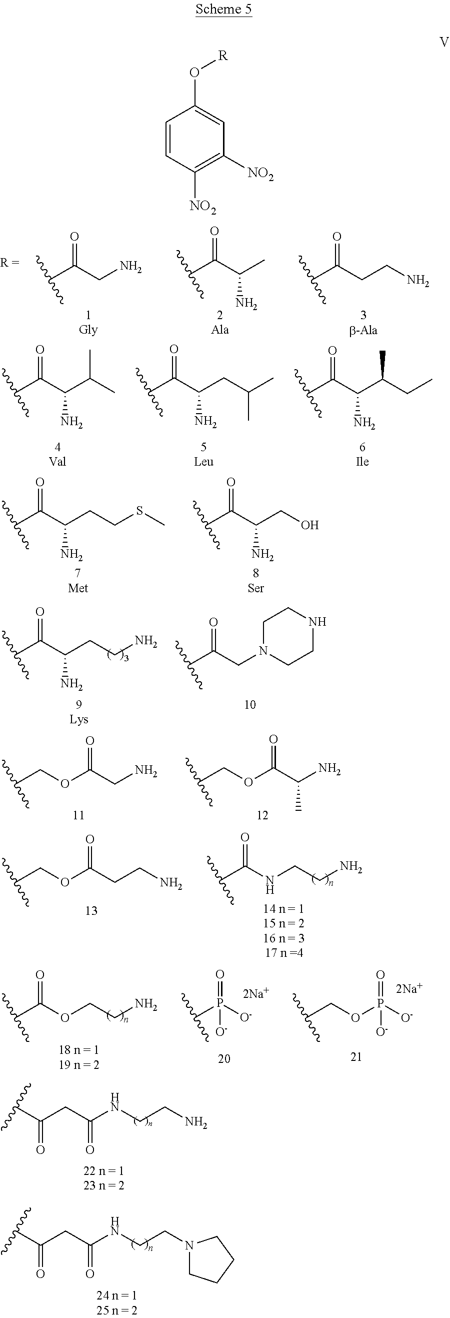

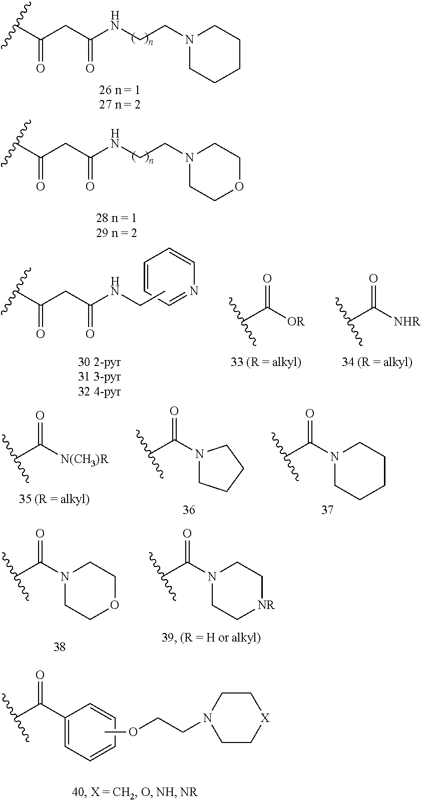

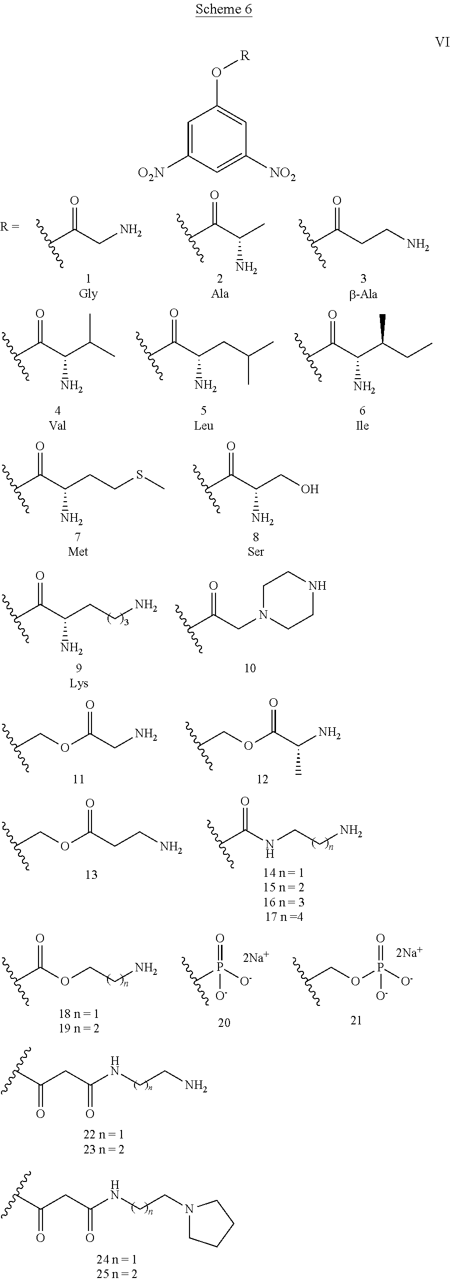

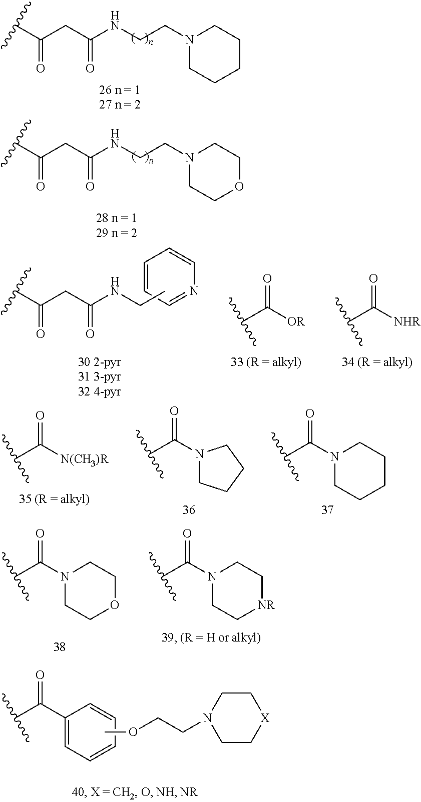

In some embodiments, a broad range of 2,3-DNP, 2,4-DNP, 2,5-DNP, 2,6-DNP, 3,4-DNP, or 3,5-DNP prodrugs can be considered. In some embodiments, the prodrugs are represented by formulas I-1 to I-40; II-1 to II-40; III-1 to III-40; IV-1 to IV-40; V-1 to V-40; and VI-1 to VI-40 are considered. A computational screen utilizing Pharma Algorithms' ADME-Tox calculator is conducted in order to identify virtual ‘hits’ (i.e. prodrugs with acceptable “predicted” oral bioavailabilities and water solubilities from the 32 prodrugs listed). The virtual screen allows ranking the 10 best prodrug molecules for synthesis and testing.

Amino acid (AA) esters of 2,3-DNP, 2,4-DNP, 2,5-DNP, 2,6-DNP, 3,4-DNP, or 3, 5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10); AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13); amino carbamates 2,3-DNP, 2,4-DNP, 2,5-DNP, 2,6-DNP, 3,4-DNP, or 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17); amino carbonates 2,3-DNP, 2,4-DNP, 2,5-DNP, 2,6-DNP, 3,4-DNP, or 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19); phosphate analogs I-20, 1-21, II-20, II-21, III-20, III-21, IV-20, IV-21, V-20, V-21, VI-20, and VI-21 (Schemes 1-6): 1,3 diketo analogs I-22 to I-32; II-22 to II-32; III-22 to III-32; IV-22 to IV-32; V-22 to V-32; and VI-22 to VI-32 (Schemes 1-6); carbonate and carbamate analogs I-33 to I-39; II-33 to II-39; III-33 to III-39; IV-33 to IV-39; V-33 to V-39; and VI-33 to VI-39 (Schemes 1-6); benzoate analogs I-40, II-40, III-40, IV-40, V-40, and VI-40 (Schemes 1-6); and combinations thereof, may be considered.

Examples of ionizable amine-containing prodrugs have increased water-solubility compared to the parent compound. In addition, AA ester prodrugs have the potential to further increase oral bioavailability due to active absorption by transporters (e.g., small peptide transporter PEPT1). For example, the valine-containing prodrugs valacyclovir and valganciclovir are substrates for the enzyme PEPT1. These AA containing prodrugs are hydrolyzed to the parent drug by aminopeptidase enzymes in the brush border membrane of the GI tract. Prodrugs which can penetrate into the peripheral circulation by passive permeation and/or by active transport are hydrolyzed by various peptidase enzymes in plasma.

Two types of phosphate prodrugs of 2,3-DNP, 2,4-DNP, 2,5-DNP, 2,6-DNP, 3,4-DNP, or 3,5-DNP of formulas I-20, I-21, II-20, II-21, III-20, III-21, IV-20, IV-21, V-20, V-21, VI-20, and VI-21 (Schemes 1-6) are synthesized. According to ADME-Tox computational data, the oral bioavailability of these prodrugs can be poor, given the fact that the prediction by ADME-Tox calculator is mainly based on the physicochemical proprieties of the molecule. Since phosphoric acid is a highly polar and extensively ionized pro-moiety, phosphate prodrugs can have significantly decreased membrane permeability compared to the parent drug.

The reasons for the success of phosphate prodrugs of 2,3-DNP, 2,4-DNP, 2,5-DNP, 2,6-DNP, 3,4-DNP, or 3,5-DNP shown in formulas I-20, I-21, II-20, II-21, III-20, III-21, IV-20, IV-21, V-20, V-21, VI-20, and VI-21 (Schemes 1-6), e.g., phosphate esters as oral prodrugs, are: 1) oral absorption is not limited by dissolution-rate, since phosphate prodrugs are highly soluble in GI tract fluids; 2) phosphate esters are chemically stable enough to prevent the precipitation of the parent drug in the GI tract; 3) phosphates are rapidly hydrolyzed by membrane-bound alkaline phosphatases, which are in abundance on the brush border surface of the cells lining the small intestine, i.e., the enterocytes. Thus, the more permeable parent drug will be released, and readily cross the enterocyte membranes and enter the systemic circulation.

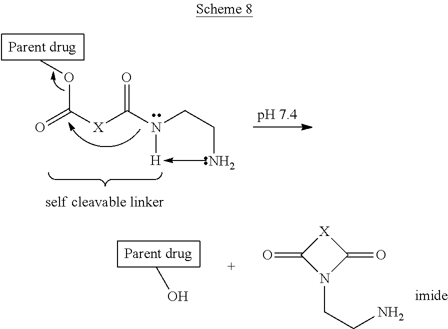

The prodrugs that are synthesized are modified to ensure water solubility. In some embodiments, acceptable solubility is tested prior to dosing in animals. If solubility is an issue, the structure of the prodrug is altered by introducing a water-soluble prodrug moiety into the molecule through conjugation with the free phenolic functionality. In other embodiments, a prodrug linker moiety that confers water-solubility properties on the prodrug molecule is utilized. In some embodiments, a solution to any water solubility issues is utilizing soft alkyl ether prodrugs that incorporate ethyleneoxy groups into promoieties such as alkyloxycarbonylmethyl (AOCOM) and N-alkyl-N-alkyloxycarbonylamino methyl (NANAOCAM) prodrugs. These prodrugs have been found to be useful for the delivery of phenolic drug molecules and have generally acceptable water solubility and membrane permeation characteristics, since they associate strongly with water molecules have good lipid solubility.

### Synthesis of “Gemini” Prodrugs of 2,3-DNP, 2,4-DNP, 2,5-DNP, 2,6-DNP, 3,4-DNP, or 3,5-DNP

In an embodiment, 2,3-DNP, 2,4-DNP, 2,5-DNP, 2,6-DNP, 3,4-DNP, or 3,5-DNP “Gemini” prodrugs may be prepared by reacting 2,3-DNP, 2,4-DNP, 2,5-DNP, 2,6-DNP, 3,4-DNP, or 3,5-DNP with triphosgene, in the presence of K₂CO₃ in dichloromethane to get 2,4-dinitrophenyl carbonochloridate, which on further reaction with bases like morpholine, piperidine, piperazine, N-alkyl piperazine yields the DNP prodrugs as illustrated in Scheme 9.

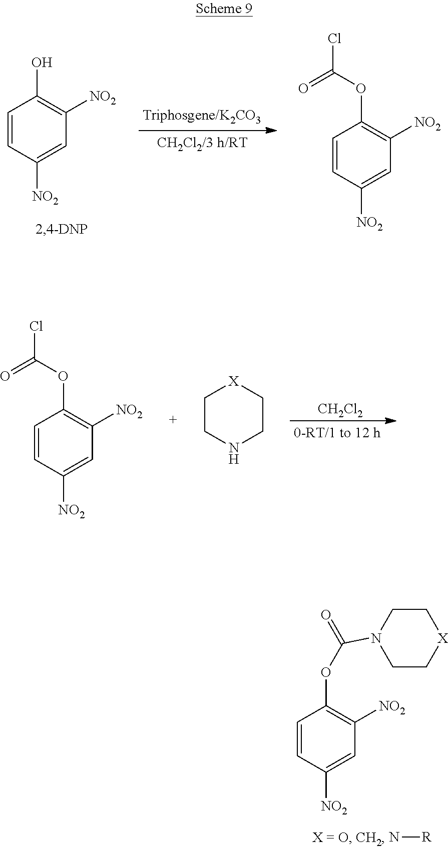

In one embodiment, 2,3-DNP, 2,4-DNP, 2,5-DNP, 2,6-DNP, 3,4-DNP, or 3,5-DNP “Gemini” prodrugs are prepared by reacting 2,3-DNP, 2,4-DNP, 2,5-DNP, 2,6-DNP, 3,4-DNP, or 3,5-DNP with 5-(tert-butyldimethylsiloxy) isophthaloyl dichloride (2) in the presence of pyridine/dichloromethane to afford precursor (3); the TBDMS protecting group will then be removed in acetone/HCl to afford prodrug (4), which will afford two equivalents of 2, 4-DNP upon hydrolysis in plasma. While the prodrug linker moiety in (4) is an ester group, other alternative linkers, such as sterically hindered ester linkers, carbonate linkers, carbamate linkers, phosphate linkers, and AOCOM and NANAOCAM based linkers may also be incorporated into the prodrug structure in order to achieve appropriate sustained release kinetics. The presence of the free phenolic group in (4) can also be utilized to improve water-solubility, if this is deemed necessary, through conjugation with appropriate hydrophilic moieties (Scheme 10).

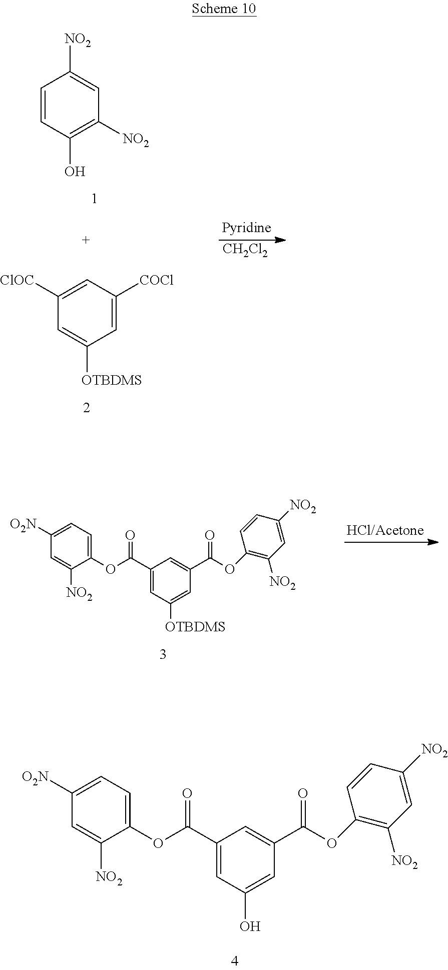

### 2,3-DNP, 2, 4-DNP, 2,5-DNP, 2,6-DNP, 3,4-DNP, or 3,5-DNP Bioprecursors

Bioprecursors of 2,3-DNP, 2,4-DNP, 2,5-DNP, 2,6-DNP, 3,4-DNP, or 3,5-DNP that may release 2,3-DNP, 2,4-DNP, 2, 5-DNP, 2,6-DNP, 3,4-DNP, or 3,5-DNP after oxidative metabolism by cytochrome P-450 may be utilized. Scheme 8 shows the design of two bioprecursors that can release 2- and 4-equivalents of 2,3-DNP, 2,4-DNP, 2,5-DNP, 2,6-DNP, 3,4-DNP, or 3,5-DNP after oxidation at the benzylic carbon by Cyt P-450 (oxidation site shown by arrows). This oxidation converts the benzylic CH₂ group into a CO group to afford an ester moiety, which can then be cleaved by esterolysis to afford 2,3-DNP, 2,4-DNP, 2,5-DNP, 2,6-DNP, 3,4-DNP, or 3,5-DNP. The use of bioprecursors is often a good alternative to the prodrug approach and may provide metabolically activated slow release of 2, 4-DNP.

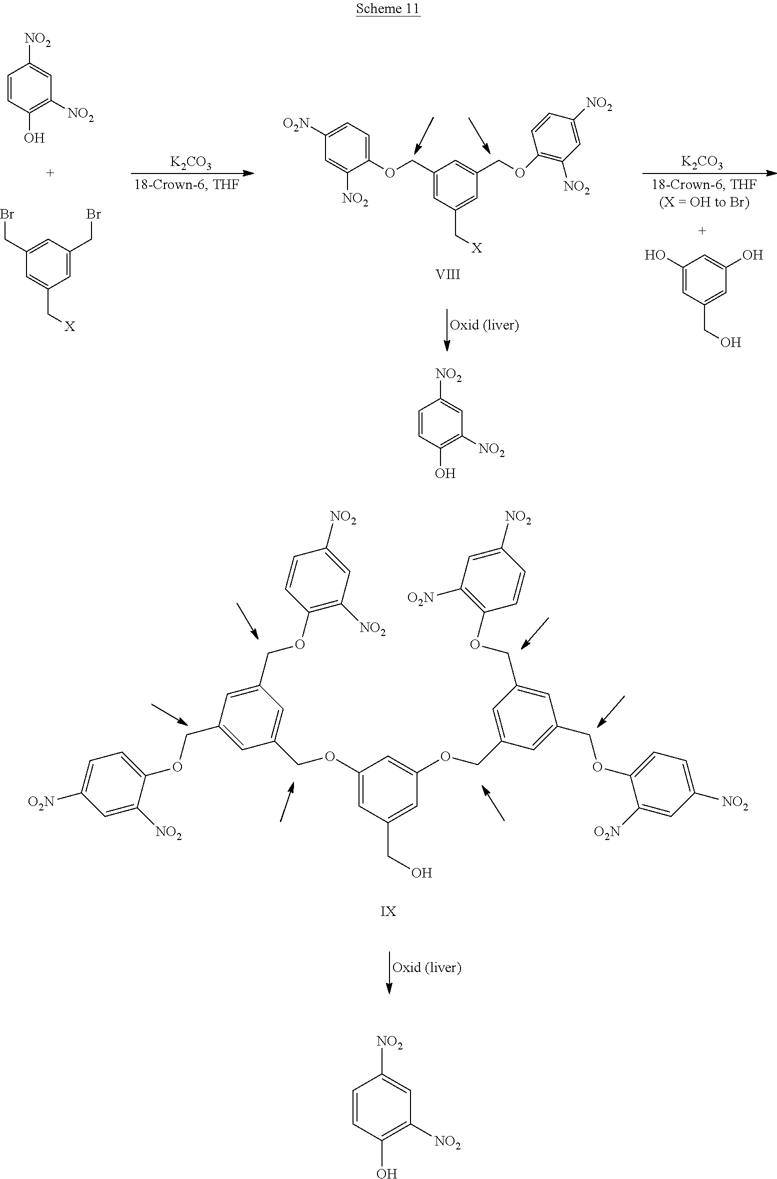

### Alternative Strategies Conjugation

In some embodiments, DNP prodrugs and bioprecursors with linkers containing open functional groups are utilized, which allow conjugation of each of these entities to nanoparticles, such as dendrimers, in order to modulate the pharmacokinetics of the molecule to enable “trickle” drug delivery. Such DNP prodrugs and bioprecursors are delivered as depot nanoparticle formulations that release DNP in a slow, sustained fashion at low doses, compared to dose and release of DNP alone, to avoid possible toxicity issues.

Nanotechnology presents an opportunity to increase the bioavailability of drug particles. A decrease in particle size results in increased surface area, results in faster dissolution. In some embodiments, the decrease is by a small order of magnitude. In other embodiments, this may be enough to result in increased bioavailability. However, faster dissolution may not be sufficient to overcome exposure to acid and enzymes in the gut. Additionally, as in the case with oral insulin, this exposure may require higher dosing of the drug, resulting in unnecessary and potentially undesirable subject exposure to breakdown products as well as create significant waste.

A depot nanoparticle formulation is specially formulated to provide slow absorption of the drug from the site of administration, often keeping therapeutic levels of the drug in the patient's system for days or weeks at a time. Alternatively, a depot nanoparticle formulation may provide convenience for a patient in need of chronic medication. By delivering drug without exposure to the GI tract, the potential issue of drug degradation is avoided. Moreover, a depot nanoparticle formulation may provide better compliance due to the infrequent dosing regimen and convenience. Additional characteristics of a depot nanoparticle formulation that will enhance patient compliance are good local tolerance at the injection site and ease of administration. Good local tolerance means minimal irritation and inflammation at the site of injection; ease of administration refers to the size of needle and length of time required to administer a dose of a particular drug formulation.

### In Vitro and In Vivo Evaluation of Prodrugs Bioprecursors

The prodrugs and bioprecursors of the disclosure encounter a wide range of pHs and enzymes when administered orally to patients. In one embodiment, the prodrug/bioprecursor is stable in the environment of the GI tract, but releases parent drug in the plasma in a sustained manner after absorption from the GI tract. Oral dosing exposes compounds to pH 1 to 2 in the stomach, pH 4.5 at the beginning of the small intestine, pH 6.6 as an average pH for the small intestine, and pHs of 5 to 9 in the colon. Stability-indicating methods are performed in aqueous buffer solutions and simulated GI fluids to determine the resilience of the 2, 3-DNP, 2,4-DNP, 2,5-DNP, 2,6-DNP, 3,4-DNP, or 3,5-DNP prodrugs/bioprecursors in the GI tract, and also their susceptibility to enzymatic conversion to the parent drug in rat plasma. These are useful methods for in vitro evaluation of the chemical stability of a prodrug candidate.

Determining pH stability in aqueous buffers (37° C., pH 1-9)
1) Determining GI stability in simulated gastric fluid (USP, 37° C.)
2) Determining GI stability in simulated intestinal fluid (USP, 37° C.)
3) Determining plasma: stability in rat plasma (37° C.)

Single compound dosing studies, are carried out on 2,3-DNP, 2,4-DNP, 2,5-DNP, 2,6-DNP, 3,4-DNP, or 3,5-DNP_prodrug/bioprecursor candidates to determine their clinical potential. Most promising preclinical prodrug/bioprecursor candidates are absorbed intact from the gastrointestinal tract and are efficiently cleaved enzymatically in plasma to afford the parent drug. The presence and identification of the prodrug and the parent drug can also provide important information on the mechanism of action of 2,3-DNP, 2,4-DNP, 2, 5-DNP, 2,6-DNP, 3,4-DNP, or 3,5-DNP, the identification of which is of value in the selection of new structural entities for consideration in structure-activity and structural optimization studies.

Pharmacokinetic studies are carried out on the most promising prodrug/bioprecursor candidate (i.e., the prodrug/bioprecursor that exhibits the greatest stability in the GI tract and affords sustained release of 2,3-DNP, 2,4-DNP, 2,5-DNP, 2,6-DNP, 3,4-DNP, or 3,5-DNP in vitro and in vivo in rat plasma) in the Sprague-Dawley rat. A complete PK profile is obtained for the orally administered prodrug in jugular and femoral vein catheterized rats to determine half-life (t_{1/2}), maximum plasma concentration (tₘₐₓ), time to reach maximum plasma concentration (tₘₐₓ), volume of distribution (Vₛₛ), area under the plasma concentration versus time curve from time 0 to infinity (AUC_{0-∞}), and bioavailability (F %), as well as other important PK parameters such as protein binding. LC/MS/MS is used as the analytical methodology to determine both the above pharmacokinetic parameters of the prodrug, and the plasma concentration and release kinetics of 2,3-DNP, 2,4-DNP, 2,5-DNP, 2,6-DNP, 3,4-DNP, or 3,5-DNP resulting from enzymatic conversion of the prodrug to 2,3-DNP, 2,4-DNP, 2,5-DNP, 2,6-DNP, 3,4-DNP, or 3,5-DNP in the plasma.

Although DNP is orally bioavailable, with good distribution and ˜6-hour half-life in rats, the prodrug approach allows extending the plasma residence time at lower concentrations of the parent drug (DNP), by appropriate design of the prodrug release characteristics.

In an embodiment, the composition for treatment of cancer and/or pain and/or pain related conditions is independently selected from the group consisting of 2,3-DNP, 2,4-DNP, 2,5-DNP, 2,6-DNP, 3,4-DNP, or 3,5-DNP, bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs; Gemini prodrugs, bioprecursor molecules, and combinations thereof.

In one embodiment, a dose of any of the foregoing embodiments of the compositions, of DNP, DNP prodrugs; Gemini prodrugs, bioprecursor molecules, and combinations thereof, for treatment of cancer and/or pain and/or pain related conditions may be from about 0.001 mg/kg to about 50 mg/kg of body weight of the patient in need of treatment: about 0.001 mg/kg to about 0.01 mg/kg of body weight of the patient in need of treatment; about 0.01 mg/kg to about 50 mg/kg of body weight of the patient in need of treatment; from about 25 mg/kg to about 100 mg/kg of body weight of the patient in need of treatment; or from about 25 mg/kg to about 100 mg/kg of body weight of the patient in need of treatment. In one embodiment, a dose of any of the foregoing embodiments of the compositions for treatment of cancer and/or pain and/or pain related conditions may be from about 0.001 mg/kg to about 50 mg/kg of body weight of the patient in need of treatment; about 0.001 mg/kg to about 0.01 mg/kg of body weight of the patient in need of treatment; 0.01 mg/kg to about 50 mg/kg of body weight of the patient in need of treatment; from 25 mg/kg to about 100 mg/kg of body weight of the patient in need of treatment; or from 25 mg/kg to about 100 mg/kg of body weight of the patient in need of treatment. In an embodiment, the present disclosure relates to a pharmaceutical composition of DNP, or a pharmaceutically acceptable salt, solvate, hydrate and/or prodrug thereof as described herein, comprising a unit dose, wherein the unit dose is in the range of about 0.1 mg to about 3000 mg. In an embodiment, the present disclosure relates to a pharmaceutical composition of DNP, or a pharmaceutically acceptable salt, solvate, hydrate and/or prodrug thereof as described herein, comprising a unit dose, wherein the unit dose is in the range of 0.1 mg to about 3000 mg.

In one embodiment, a dose of any of the foregoing embodiments of the compositions, of DNP, DNP prodrugs; Gemini prodrugs, bioprecursor molecules, and combinations thereof, for treatment of cancer including breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, may be independently selected from about 0.001 mg/kg of body weight to about 50 mg/kg of body weight; about 0.001 mg/kg to about 50 mg/kg of body weight; about 0.001 mg/kg to about 40 mg/kg of body weight; about 0.001 mg/kg to about 30 mg/kg of body weight; about 0.001 mg/kg to about 20 mg/kg of body weight; about 0.001 mg/kg of body weight to about 15 mg/kg of body weight; about 0.001 mg/kg to about 10 mg/kg of body weight; about 0.001 mg/kg to about 5 mg/kg of body weight; about 0.001 mg/kg to about 1 mg/kg of body weight; about 0.001 mg/kg of body weight to about 0.1 mg/kg of body weight; about 0.001 mg/kg of body weight to about 0.01 mg/kg of body weight; about 0.01 mg/kg to about 50 mg/kg of body weight; about 0.01 mg/kg to about 40 mg/kg of body weight; about 0.01 mg/kg to about 30 mg/kg of body weight; about 0.01 mg/kg to about 20 mg/kg of body weight; about 0.01 mg/kg of body weight to about 15 mg/kg of body weight; about 0.01 mg/kg to about 10 mg/kg of body weight; about 0.01 mg/kg to about 5 mg/kg of body weight; about 0.01 mg/kg to about 1 mg/kg of body weight; about 0.05 mg/kg to about 50 mg/kg of body weight; about 0.05 mg/kg to about 40 mg/kg of body weight; about 0.05 mg/kg to about 30 mg/kg of body weight; about 0.05 mg/kg to about 20 mg/kg of body weight; about 0.05 mg/kg to about 10 mg/kg of body weight; about 0.05 mg/kg to about 1.0 mg/kg of body weight; about 0.05 mg/kg to about 0.1 mg/kg of body weight; about 0.1 mg/kg to about 40 mg/kg of body weight; about 0.1 mg/kg to about 50 mg/kg of body weight; about 0.1 mg/kg to about 30 mg/kg of body weight; about 0.1 mg/kg to about 20 mg/kg of body weight; about 0.1 mg/kg to about 15 mg/kg of body weight; about 0.1 mg/kg to about 12 mg/kg of body weight; about 0.1 mg/kg to about 10 mg/kg of body weight; about 0.1 mg/kg to about 9 mg/kg of body weight; about 0.1 mg/kg to about 8 mg/kg of body weight; about 0.1 mg/kg to about 7 mg/kg of body weight; about 0.1 mg/kg to about 6 mg/kg of body weight; about 0.1 mg/kg to about 5 mg/kg of body weight; about 0.1 mg/kg to about 4 mg/kg of body weight; about 0.1 mg/kg to about 3 mg/kg of body weight; about 0.1 mg/kg to about 2 mg/kg of body weight; 0.1 mg/kg to about 1.0 mg/kg of body weight; about 0.3 mg/kg to about 20 mg/kg of body weight; about 0.3 mg/kg to about 15 mg/kg of body weight; about 0.3 mg/kg to about 12 mg/kg of body weight; about 0.3 mg/kg to about 10 mg/kg of body weight; about 0.3 mg/kg to about 9 mg/kg of body weight; about 0.3 mg/kg to about 8 mg/kg of body weight; about 0.3 mg/kg to about 7 mg/kg of body weight; about 0.3 mg/kg to about 6 mg/kg of body weight; about 0.3 mg/kg to about 5 mg/kg of body weight; about 0.3 mg/kg to about 4 mg/kg of body weight; about 0.3 mg/kg to about 3 mg/kg of body weight; about 0.3 mg/kg to about 2 mg/kg of body weight; about 0.3 mg/kg to about 1.0 mg/kg of body weight; about 0.5 mg/kg to about 15 mg/kg of body weight; about 0.5 mg/kg to about 12 mg/kg of body weight; about 0.5 mg/kg to about 10 mg/kg of body weight; about 0.5 mg/kg to about 9 mg/kg of body weight; about 0.5 mg/kg to about 8 mg/kg of body weight; about 0.5 mg/kg to about 7 mg/kg of body weight; about 0.5 mg/kg to about 6 mg/kg of body weight; about 0.5 mg/kg to about 5 mg/kg of body weight; about 0.5 mg/kg to about 4 mg/kg of body weight; about 0.5 mg/kg to about 3 mg/kg of body weight; about 0.5 mg/kg to about 2 mg/kg of body weight; about 0.5 mg/kg to about 1.0 mg/kg of body weight; about 0.8 mg/kg to about 15 mg/kg of body weight; about 0.8 mg/kg to about 12 mg/kg of body weight; about 0.8 mg/kg to about 10 mg/kg of body weight; about 0.8 mg/kg to about 9 mg/kg of body weight; about 0.8 mg/kg to about 8 mg/kg of body weight; about 0.8 mg/kg to about 7 mg/kg of body weight; about 0.8 mg/kg to about 6 mg/kg of body weight; about 0.8 mg/kg to about 5 mg/kg of body weight; about 0.8 mg/kg to about 4 mg/kg of body weight; about 0.8 mg/kg to about 3 mg/kg of body weight; about 0.8 mg/kg to about 2 mg/kg of body weight; about 0.8 mg/kg to about 1.0 mg/kg of body weight; about 1 mg/kg to about 3.0 mg/kg of body; about 1.5 mg/kg to about 3.0 mg/kg of body; about 1.0 mg/kg to about 2.0 mg/kg of body; about 2.0 mg/kg to about 3.0 mg/kg of body; about 0.5 mg/kg to about 2.5 mg/kg of body weight; about 0.5 mg/kg to about 2.0 mg/kg of body weight. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer.

In one embodiment, a dose of any of the foregoing embodiments of the compositions, of DNP, DNP prodrugs; Gemini prodrugs, bioprecursor molecules, and combinations thereof, for treatment of pain and/or a pain related conditions including pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition, may be independently selected from about 0.001 mg/kg of body weight to about 50 mg/kg of body weight; about 0.001 mg/kg to about 50 mg/kg of body weight; about 0.001 mg/kg to about 40 mg/kg of body weight; about 0.001 mg/kg to about 30 mg/kg of body weight; about 0.001 mg/kg to about 20 mg/kg of body weight; about 0.001 mg/kg of body weight to about 15 mg/kg of body weight; about 0.001 mg/kg to about 10 mg/kg of body weight; about 0.001 mg/kg to about 5 mg/kg of body weight; about 0.001 mg/kg to about 1 mg/kg of body weight; about 0.001 mg/kg of body weight to about 0.1 mg/kg of body weight; about 0.001 mg/kg of body weight to about 0.01 mg/kg of body weight; about 0.01 mg/kg to about 50 mg/kg of body weight: about 0.01 mg/kg to about 40 mg/kg of body weight; about 0.01 mg/kg to about 30 mg/kg of body weight; about 0.01 mg/kg to about 20 mg/kg of body weight; about 0.01 mg/kg of body weight to about 15 mg/kg of body weight; about 0.01 mg/kg to about 10 mg/kg of body weight; about 0.01 mg/kg to about 5 mg/kg of body weight; about 0.01 mg/kg to about 1 mg/kg of body weight; about 0.05 mg/kg to about 50 mg/kg of body weight; about 0.05 mg/kg to about 40 mg/kg of body weight; about 0.05 mg/kg to about 30 mg/kg of body weight; about 0.05 mg/kg to about 20 mg/kg of body weight; about 0.05 mg/kg to about 10 mg/kg of body weight; about 0.05 mg/kg to about 1.0 mg/kg of body weight; about 0.05 mg/kg to about 0.1 mg/kg of body weight; about 0.1 mg/kg to about 40 mg/kg of body weight; about 0.1 mg/kg to about 50 mg/kg of body weight; about 0.1 mg/kg to about 30 mg/kg of body weight; about 0.1 mg/kg to about 20 mg/kg of body weight; about 0.1 mg/kg to about 15 mg/kg of body weight; about 0.1 mg/kg to about 12 mg/kg of body weight; about 0.1 mg/kg to about 10 mg/kg of body weight; about 0.1 mg/kg to about 9 mg/kg of body weight; about 0.1 mg/kg to about 8 mg/kg of body weight; about 0.1 mg/kg to about 7 mg/kg of body weight; about 0.1 mg/kg to about 6 mg/kg of body weight; about 0.1 mg/kg to about 5 mg/kg of body weight; about 0.1 mg/kg to about 4 mg/kg of body weight; about 0.1 mg/kg to about 3 mg/kg of body weight; about 0.1 mg/kg to about 2 mg/kg of body weight; 0.1 mg/kg to about 1.0 mg/kg of body weight; about 0.3 mg/kg to about 20 mg/kg of body weight; about 0.3 mg/kg to about 15 mg/kg of body weight; about 0.3 mg/kg to about 12 mg/kg of body weight; about 0.3 mg/kg to about 10 mg/kg of body weight; about 0.3 mg/kg to about 9 mg/kg of body weight; about 0.3 mg/kg to about 8 mg/kg of body weight; about 0.3 mg/kg to about 7 mg/kg of body weight; about 0.3 mg/kg to about 6 mg/kg of body weight; about 0.3 mg/kg to about 5 mg/kg of body weight; about 0.3 mg/kg to about 4 mg/kg of body weight; about 0.3 mg/kg to about 3 mg/kg of body weight; about 0.3 mg/kg to about 2 mg/kg of body weight; about 0.3 mg/kg to about 1.0 mg/kg of body weight; about 0.5 mg/kg to about 15 mg/kg of body weight; about 0.5 mg/kg to about 12 mg/kg of body weight; about 0.5 mg/kg to about 10 mg/kg of body weight; about 0.5 mg/kg to about 9 mg/kg of body weight; about 0.5 mg/kg to about 8 mg/kg of body weight; about 0.5 mg/kg to about 7 mg/kg of body weight; about 0.5 mg/kg to about 6 mg/kg of body weight; about 0.5 mg/kg to about 5 mg/kg of body weight; about 0.5 mg/kg to about 4 mg/kg of body weight; about 0.5 mg/kg to about 3 mg/kg of body weight; about 0.5 mg/kg to about 2 mg/kg of body weight; about 0.5 mg/kg to about 1.0 mg/kg of body weight; about 0.8 mg/kg to about 15 mg/kg of body weight; about 0.8 mg/kg to about 12 mg/kg of body weight; about 0.8 mg/kg to about 10 mg/kg of body weight; about 0.8 mg/kg to about 9 mg/kg of body weight; about 0.8 mg/kg to about 8 mg/kg of body weight; about 0.8 mg/kg to about 7 mg/kg of body weight; about 0.8 mg/kg to about 6 mg/kg of body weight; about 0.8 mg/kg to about 5 mg/kg of body weight; about 0.8 mg/kg to about 4 mg/kg of body weight; about 0.8 mg/kg to about 3 mg/kg of body weight; about 0.8 mg/kg to about 2 mg/kg of body weight; about 0.8 mg/kg to about 1.0 mg/kg of body weight; about 1 mg/kg to about 3.0 mg/kg of body: about 1.5 mg/kg to about 3.0 mg/kg of body; about 1.0 mg/kg to about 2.0 mg/kg of body; about 2.0 mg/kg to about 3.0 mg/kgof body; about 0.5 mg/kg to about 2.5 mg/kg of body weight; about 0.5 mg/kg to about 2.0 mg/kg of body weight. In some embodiments, the pain and/or pain related condition is selected from acute pain, chronic pain, neuropathic pain, nociceptive pain, and radicular pain.

In one embodiment, a dose of any of the foregoing embodiments of the compositions, of DNP, DNP prodrugs; Gemini prodrugs, bioprecursor molecules, and combinations thereof, for treatment of cancer including breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, may be independently selected from about 0.001 mg/kg of body weight to about 50 mg/kg of body weight; about 0.001 mg/kg to about 50 mg/kg of body weight; about 0.001 mg/kg to about 40 mg/kg of body weight; about 0.001 mg/kg to about 30 mg/kg of body weight; about 0.001 mg/kg to about 20 mg/kg of body weight; about 0.001 mg/kg of body weight to about 15 mg/kg of body weight; about 0.001 mg/kg to about 10 mg/kg of body weight; about 0.001 mg/kg to about 5 mg/kg of body weight; about 0.001 mg/kg to about 1 mg/kg of body weight; about 0.001 mg/kg of body weight to about 0.1 mg/kg of body weight; about 0.001 mg/kg of body weight to about 0.01 mg/kg of body weight; about 0.01 mg/kg to about 50 mg/kg of body weight; about 0.01 mg/kg to about 40 mg/kg of body weight; about 0.01 mg/kg to about 30 mg/kg of body weight; about 0.01 mg/kg to about 20 mg/kg of body weight; about 0.01 mg/kg of body weight to about 15 mg/kg of body weight; about 0.01 mg/kg to about 10 mg/kg of body weight; about 0.01 mg/kg to about 5 mg/kg of body weight; about 0.01 mg/kg to about 1 mg/kg of body weight; about 0.05 mg/kg to about 50 mg/kg of body weight; about 0.05 mg/kg to about 40 mg/kg of body weight; about 0.05 mg/kg to about 30 mg/kg of body weight; about 0.05 mg/kg to about 20 mg/kg of body weight; about 0.05 mg/kg to about 10 mg/kg of body weight; about 0.05 mg/kg to about 1.0 mg/kg of body weight; about 0.05 mg/kg to about 0.1 mg/kg of body weight; about 0.1 mg/kg to about 40 mg/kg of body weight; about 0.1 mg/kg to about 50 mg/kg of body weight; about 0.1 mg/kg to about 30 mg/kg of body weight; about 0.1 mg/kg to about 20 mg/kg of body weight; about 0.1 mg/kg to about 15 mg/kg of body weight; about 0.1 mg/kg to about 12 mg/kg of body weight; about 0.1 mg/kg to about 10 mg/kg of body weight; about 0.1 mg/kg to about 9 mg/kg of body weight; about 0.1 mg/kg to about 8 mg/kg of body weight; about 0.1 mg/kg to about 7 mg/kg of body weight; about 0.1 mg/kg to about 6 mg/kg of body weight; about 0.1 mg/kg to about 5 mg/kg of body weight; about 0.1 mg/kg to about 4 mg/kg of body weight; about 0.1 mg/kg to about 3 mg/kg of body weight; about 0.1 mg/kg to about 2 mg/kg of body weight; about 0.1 mg/kg to about 1.0 mg/kg of body weight; about 0.3 mg/kg to about 20 mg/kg of body weight; about 0.3 mg/kg to about 15 mg/kg of body weight; about 0.3 mg/kg to about 12 mg/kg of body weight; about 0.3 mg/kg to about 10 mg/kg of body weight; about 0.3 mg/kg to about 9 mg/kg of body weight; about 0.3 mg/kg to about 8 mg/kg of body weight; about 0.3 mg/kg to about 7 mg/kg of body weight; about 0.3 mg/kg to about 6 mg/kg of body weight; about 0.3 mg/kg to about 5 mg/kg of body weight; about 0.3 mg/kg to about 4 mg/kg of body weight; about 0.3 mg/kg to about 3 mg/kg of body weight; about 0.3 mg/kg to about 2 mg/kg of body weight; about 0.3 mg/kg to about 1.0 mg/kg of body weight; about 0.5 mg/kg to about 10 mg/kg of body weight; about 0.5 mg/kg to about 9 mg/kg of body weight; about 0.5 mg/kg to about 8 mg/kg of body weight; about 0.5 mg/kg to about 7 mg/kg of body weight; about 0.5 mg/kg to about 6 mg/kg of body weight; about 0.5 mg/kg to about 5 mg/kg of body weight; about 0.5 mg/kg to about 4 mg/kg of body weight; about 0.5 mg/kg to about 3 mg/kg of body weight; about 0.5 mg/kg to about 2 mg/kg of body weight; about 0.5 mg/kg to about 1.0 mg/kg of body weight; about 0.8 mg/kg to about 15 mg/kg of body weight; about 0.8 mg/kg to about 12 mg/kg of body weight; about 0.8 mg/kg to about 8 10 mg/kg of body weight; about 0.8 mg/kg to about 9 mg/kg of body weight; about 0.8 mg/kg to about 8 mg/kg of body weight; about 0.8 mg/kg to about 7 mg/kg of body weight; about 0.8 mg/kg to about 6 mg/kg of body weight; about 0.8 mg/kg to about 5 mg/kg of body weight; about 0.8 mg/kg to about 4 mg/kg of body weight; about 0.8 mg/kg to about 3 mg/kg of body weight; about 0.8 mg/kg to about 2 mg/kg of body weight; about 0.8 mg/kg to about 1.0 mg/kg of body weight; about 1 mg/kg to about 3.0 mg/kg of body; about 1.5 mg/kg to about 3.0 mg/kg of body; about 1.0 mg/kg to about 2.0 mg/kg of body; about 2.0 mg/kg to about 3.0 mg/kg of body; about 0.5 mg/kg to about 2.5 mg/kg of body weight; about 0.5 mg/kg to about 2.0 mg/kg of body weight, or about 0.001 mg/kg of body weight, about 0.002 mg/kg of body weight, about 0.003 mg/kg of body weight, about 0.004 mg/kg of body weight, about 0.005 mg/kg of body weight, about 0.006 mg/kg of body weight, about 0.007 mg/kg of body weight, about 0.008 mg/kg of body weight, about 0.009 mg/kg of body weight, about 0.01 mg/kg of body weight, about 0.02 mg/kg of body weight, about 0.03 mg/kg of body weight, about 0.04 mg/kg of body weight, about 0.05 mg/kg of body weight, about 0.06 mg/kg of body weight, about 0.07 mg/kg of body weight, about 0.08 mg/kg of body weight, about 0.09 mg/kg of body weight, about 0.1 mg/kg of body weight, about 0.2 mg/kg of body weight, about 0.3 mg/kg of body weight, about 0.4 mg/kg of body weight, about 0.5 mg/kg of body weight, about 0.6 mg/kg of body weight, about 0.7 mg/kg of body weight, about 0.8 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, or about 20 mg/kg of body weight. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer.

In one embodiment, a dose of any of the foregoing embodiments of the compositions, of DNP, DNP prodrugs; Gemini prodrugs, bioprecursor molecules, and combinations thereof, for treatment of pain and/or a pain related conditions including pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition, may be independently selected from about 0.001 mg/kg of body weight to about 50 mg/kg of body weight; about 0.001 mg/kg to about 50 mg/kg of body weight; about 0.001 mg/kg to about 40 mg/kg of body weight; about 0.001 mg/kg to about 30 mg/kg of body weight; about 0.001 mg/kg to about 20 mg/kg of body weight; about 0.001 mg/kg of body weight to about 15 mg/kg of body weight; about 0.001 mg/kg to about 10 mg/kg of body weight; about 0.001 mg/kg to about 5 mg/kg of body weight; about 0.001 mg/kg to about 1 mg/kg of body weight; about 0.001 mg/kg of body weight to about 0.1 mg/kg of body weight; about 0.001 mg/kg of body weight to about 0.01 mg/kg of body weight; about 0.01 mg/kg to about 50 mg/kg of body weight: about 0.01 mg/kg to about 40 mg/kg of body weight; about 0.01 mg/kg to about 30 mg/kg of body weight; about 0.01 mg/kg to about 20 mg/kg of body weight; about 0.01 mg/kg to about 10 mg/kg of body weight; about 0.01 mg/kg to about 5 mg/kg of body weight; about 0.01 mg/kg to about 1 mg/kg of body weight; about 0.05 mg/kg to about 50 mg/kg of body weight; about 0.05 mg/kg to about 40 mg/kg of body weight; about 0.05 mg/kg to about 30 mg/kg of body weight; about 0.05 mg/kg to about 20 mg/kg of body weight; about 0.05 mg/kg to about 10 mg/kg of body weight; about 0.05 mg/kg to about 1.0 mg/kg of body weight; about 0.05 mg/kg to about 0.1 mg/kg of body weight; about 0.1 mg/kg to about 40 mg/kg of body weight; about 0.1 mg/kg to about 50 mg/kg of body weight; about 0.1 mg/kg to about 30 mg/kg of body weight; about 0.1 mg/kg to about 20 mg/kg of body weight; about 0.1 mg/kg to about 15 mg/kg of body weight; about 0.1 mg/kg to about 12 mg/kg of body weight; about 0.1 mg/kg to about 10 mg/kg of body weight; about 0.1 mg/kg to about 9 mg/kg of body weight; about 0.1 mg/kg to about 8 mg/kg of body weight; about 0.1 mg/kg to about 7 mg/kg of body weight; about 0.1 mg/kg to about 6 mg/kg of body weight; about 0.1 mg/kg to about 5 mg/kg of body weight; about 0.1 mg/kg to about 4 mg/kg of body weight; about 0.1 mg/kg to about 3 mg/kg of body weight; about 0.1 mg/kg to about 2 mg/kg of body weight; about 0.1 mg/kg to about 1.0 mg/kg of body weight: about 0.3 mg/kg to about 20 mg/kg of body weight; about 0.3 mg/kg to about 15 mg/kg of body weight; about 0.3 mg/kg to about 12 mg/kg of body weight; about 0.3 mg/kg to about 10 mg/kg of body weight; about 0.3 mg/kg to about 9 mg/kg of body weight; about 0.3 mg/kg to about 8 mg/kg of body weight; about 0.3 mg/kg to about 7 mg/kg of body weight; about 0.3 mg/kg to about 6 mg/kg of body weight; about 0.3 mg/kg to about 5 mg/kg of body weight; about 0.3 mg/kg to about 4 mg/kg of body weight; about 0.3 mg/kg to about 3 mg/kg of body weight; about 0.3 mg/kg to about 2 mg/kg of body weight; about 0.3 mg/kg to about 1.0 mg/kg of body weight; about 0.5 mg/kg to about 10 mg/kg of body weight; about 0.5 mg/kg to about 9 mg/kg of body weight; about 0.5 mg/kg to about 8 mg/kg of body weight; about 0.5 mg/kg to about 7 mg/kg of body weight; about 0.5 mg/kg to about 6 mg/kg of body weight; about 0.5 mg/kg to about 5 mg/kg of body weight; about 0.5 mg/kg to about 4 mg/kg of body weight; about 0.5 mg/kg to about 3 mg/kg of body weight; about 0.5 mg/kg to about 2 mg/kg of body weight; about 0.5 mg/kg to about 1.0 mg/kg of body weight; about 0.8 mg/kg to about 15 mg/kg of body weight; about 0.8 mg/kg to about 12 mg/kg of body weight; about 0.8 mg/kg to about 8 10 mg/kg of body weight; about 0.8 mg/kg to about 9 mg/kg of body weight; about 0.8 mg/kg to about 8 mg/kg of body weight; about 0.8 mg/kg to about 7 mg/kg of body weight; about 0.8 mg/kg to about 6 mg/kg of body weight; about 0.8 mg/kg to about 5 mg/kg of body weight; about 0.8 mg/kg to about 4 mg/kg of body weight; about 0.8 mg/kg to about 3 mg/kg of body weight; about 0.8 mg/kg to about 2 mg/kg of body weight; about 0.8 mg/kg to about 1.0 mg/kg of body weight; about 1 mg/kg to about 3.0 mg/kg of body; about 1.5 mg/kg to about 3.0 mg/kg of body; about 1.0 mg/kg to about 2.0 mg/kg of body; about 2.0 mg/kg to about 3.0 mg/kg of body; about 0.5 mg/kg to about 2.5 mg/kg of body weight; about 0.5 mg/kg to about 2.0 mg/kg of body weight, or about 0.001 mg/kg of body weight, about 0.002 mg/kg of body weight, about 0.003 mg/kg of body weight, about 0.004 mg/kg of body weight, about 0.005 mg/kg of body weight, about 0.006 mg/kg of body weight, about 0.007 mg/kg of body weight, about 0.008 mg/kg of body weight, about 0.009 mg/kg of body weight, about 0.01 mg/kg of body weight, about 0.02 mg/kg of body weight, about 0.03 mg/kg of body weight, about 0.04 mg/kg of body weight, about 0.05 mg/kg of body weight, about 0.06 mg/kg of body weight, about 0.07 mg/kg of body weight, about 0.08 mg/kg of body weight, about 0.09 mg/kg of body weight, about 0.1 mg/kg of body weight, about 0.2 mg/kg of body weight, about 0.3 mg/kg of body weight, about 0.4 mg/kg of body weight, about 0.5 mg/kg of body weight, about 0.6 mg/kg of body weight, about 0.7 mg/kg of body weight, about 0.8 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, or about 20 mg/kg of body weight. In some embodiments, the pain and/or pain related condition is selected from acute pain, chronic pain, neuropathic pain, nociceptive pain, and radicular pain.

In one embodiment, a dose of any of the foregoing embodiments of the compositions for treatment of cancer including breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, may be independently from about 0.1 mg/day/70 kg of body weight to about 300 mg/day/70 kg of body weight of the patient in need of treatment: about 1 mg/day/70 kg of body weight to about 300 mg/day/70 kg of body weight of the patient in need of treatment; about 1 mg/day/70 kg of body weight to about 200 mg/day/70 kg of body weight of the patient in need of treatment; about 1 mg/day/70 kg of body weight to about 100 mg/day/70 kg of body weight of the patient in need of treatment; about 1 mg/day/70 kg of body weight to about 50 mg/day/70 kg of body weight of the patient in need of treatment; about 1 mg/day/70 kg of body weight to about 30 mg/day/70 kg of body weight of the patient in need of treatment: about 1 mg/day/70 kg of body weight to about 20 mg/day/70 kg of body weight of the patient in need of treatment; about 1 mg/day/70 kg of body weight to about 10 mg/day/70 kg of body weight of the patient in need of treatment; about 1 mg/day/70 kg of body weight to about 5 mg/day/70 kg of body weight of the patient in need of treatment; about 0.1 mg/day/70 kg of body weight to about 10 mg/day/70 kg of body weight of the patient in need of treatment; about 0.6 mg/day/70 kg of body weight to about 6 mg/day/70 kg of body weight of the patient in need of treatment. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer.

In one embodiment, a dose of any of the foregoing embodiments of the compositions for treatment of pain and/or a pain related condition including pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition, may be independently from about 1 mg/day/70 kg of body weight to about 300 mg/day/70 kg of body weight of the patient in need of treatment; about 1 mg/day/70 kg of body weight to about 200 mg/day/70 kg of body weight of the patient in need of treatment: about 1 mg/day/70 kg of body weight to about 100 mg/day/70 kg of body weight of the patient in need of treatment; about 1 mg/day/70 kg of body weight to about 50 mg/day/70 kg of body weight of the patient in need of treatment; about 1 mg/day/70 kg of body weight to about 30 mg/day/70 kg of body weight of the patient in need of treatment; about 1 mg/day/70 kg of body weight to about 20 mg/day/70 kg of body weight of the patient in need of treatment; about 1 mg/day/70 kg of body weight to about 10 mg/day/70 kg of body weight of the patient in need of treatment; about 1 mg/day/70 kg of body weight to about 5 mg/day/70 kg of body weight of the patient in need of treatment. In some embodiments, the pain and/or pain related condition is selected from acute pain, chronic pain, neuropathic pain, nociceptive pain, and radicular pain.

In one embodiment, a dose of any of the foregoing embodiments of the compositions for treatment of cancer including breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, may be independently from 1 mg/day/70 kg of body weight to 300 mg/day/70 kg of body weight of the patient in need of treatment; 1 mg/day/70 kg of body weight to 200 mg/day/70 kg of body weight of the patient in need of treatment; 1 mg/day/70 kg of body weight to 100 mg/day/70 kg of body weight of the patient in need of treatment; 1 mg/day/70 kg of body weight to 50 mg/day/70 kg of body weight of the patient in need of treatment; 1 mg/day/70 kg of body weight to 30 mg/day/70 kg of body weight of the patient in need of treatment; 1 mg/day/70 kg of body weight to 20 mg/day/70 kg of body weight of the patient in need of treatment; 1 mg/day/70 kg of body weight to 10 mg/day/70 kg of body weight of the patient in need of treatment; 1 mg/day/70 kg of body weight to 5 mg/day/70 kg of body weight of the patient in need of treatment. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer.

In one embodiment, a dose of any of the foregoing embodiments of the compositions for treatment of pain and/or a pain related condition including pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition, may be independently from about 1 mg/day/70 kg of body weight to about 300 mg/day/70 kg of body weight of the patient in need of treatment; about 1 mg/day/70 kg of body weight to about 200 mg/day/70 kg of body weight of the patient in need of treatment; about 1 mg/day/70 kg of body weight to about 100 mg/day/70 kg of body weight of the patient in need of treatment; about 1 mg/day/70 kg of body weight to about 50 mg/day/70 kg of body weight of the patient in need of treatment; about 1 mg/day/70 kg of body weight to about 30 mg/day/70 kg of body weight of the patient in need of treatment; about 1 mg/day/70 kg of body weight to about 20 mg/day/70 kg of body weight of the patient in need of treatment; about 1 mg/day/70 kg of body weight to about 10 mg/day/70 kg of body weight of the patient in need of treatment; about 1 mg/day/70 kg of body weight to about 5 mg/day/70 kg of body weight of the patient in need of treatment; about 0.1 mg/day/70 kg of body weight to about 10 mg/day/70 kg of body weight of the patient in need of treatment; about 0.6 mg/day/70 kg of body weight to about 6 mg/day/70 kg of body weight of the patient in need of treatment. In some embodiments, the pain and/or pain related condition is selected from acute pain, chronic pain, neuropathic pain, nociceptive pain, and radicular pain.

In some embodiments, a pharmaceutical composition includes DNP, selected from the group consisting of DNP, 2,3-DNP, 2,4-DNP, 2,5-DNP, 2,6-DNP, 3,4-DNP, or 3,5-DNP, bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs or a pharmaceutically acceptable salt, solvate, or hydrate thereof, comprising a unit dose, independently selected from: wherein the unit dose is in the range of about 0.1 mg to about 3000 mg; wherein the unit dose is in the range of about 0.1 mg to about 1000 mg; wherein the unit dose is in the range of about 0.1 mg to about 500 mg; wherein the unit dose is in the range of about 0.1 mg to about 100 mg; wherein the unit dose is in the range of about 1 mg to about 50 mg; wherein the unit dose is in the range of about 0.1 mg to about 10 mg; wherein the unit dose is in the range of about 0.5 mg to about 10 mg; wherein the unit dose is in the range of about 0.6 mg to about 6 mg; wherein the unit dose is about 0.1 mg; wherein the unit dose is about 0.2 mg; wherein the unit dose is about 0.3 mg; wherein the unit dose is about 0.4 mg; wherein the unit dose is about 0.5 mg; wherein the unit dose is about 0.6 mg; wherein the unit dose is about 0.7 mg; wherein the unit dose is about 0.8 mg; wherein the unit dose is about 0.9 mg; wherein the unit dose is about 1 mg; wherein the unit dose is about 2 mg; wherein the unit dose is about 3 mg; wherein the unit dose is about 4 mg; wherein the unit dose is about 5 mg; wherein the unit dose is the range of about 5 mg to about 10 mg; wherein the unit dose is about 6 mg; wherein the unit dose is about 7 mg; wherein the unit dose is about 8 mg; wherein the unit dose is about 9 mg; wherein the unit dose is about 10 mg; wherein the unit dose is the range of about 10 mg to about 15 mg; wherein the unit dose is about 11 mg; wherein the unit dose is about 12 mg; wherein the unit dose is about 13 mg; wherein the unit dose is about 14 mg; wherein the unit dose is about 15 mg; wherein the unit dose is the range of about 15 mg to about 20 mg; wherein the unit dose is about 16 mg; wherein the unit dose is about 17 mg; wherein the unit dose is about 18 mg; wherein the unit dose is about 19 mg, wherein the unit dose is about 20 mg; wherein the unit dose is the range of about 20 mg to about 30 mg; wherein the unit dose is about 25 mg; wherein the unit dose is about 30 mg; wherein the unit dose is the range of about 30 mg to about 40 mg; wherein the unit dose is about 35 mg; wherein the unit dose is about 40 mg; wherein the unit dose is the range of about 40 mg to about 50 mg; wherein the unit dose is about 45 mg; wherein the unit dose is about 50 mg; wherein the unit dose is the range of about 50 mg to about 100 mg; wherein the unit dose is about 75 mg; wherein the unit dose is about 100 mg; wherein the unit dose is the range of about 100 mg to about 200 mg; wherein the unit dose is about 150 mg; wherein the unit dose is about 200 mg; wherein the unit dose is the range of about 200 mg to about 300 mg; wherein the unit dose is about 200 mg; wherein the unit dose is about 250 mg; wherein the unit dose is about 300 mg; wherein the unit dose is about 350 mg; wherein the unit dose is about 400 mg; wherein the unit dose is about 450 mg; wherein the unit dose is about 500 mg; wherein the unit dose is about 750 mg; wherein the unit dose is about 1000 mg; wherein the unit dose is about 1500 mg; wherein the unit dose is about 2000 mg; wherein the unit dose is about 2500 mg; or wherein the unit dose is about 3000 mg.

In some embodiments, a pharmaceutical composition includes DNP, selected from the group consisting of DNP, 2,3-DNP, 2,4-DNP, 2,5-DNP, 2,6-DNP, 3,4-DNP, or 3,5-DNP, bipartite 2,3-dinitrophenol, 2,4-dinitrophenol, 2,5-dinitrophenol, 2,6-dinitrophenol, 3,4-dinitrophenol, or 3,5-dinitrophenol (2,3-DNP, 2,4-DNP, 2,5-DNP, 2,6-DNP, 3,4-DNP, or 3,5-DNP) prodrugs or a pharmaceutically acceptable salt, solvate, or hydrate thereof, comprising a unit dose, independently selected from: wherein the unit dose is from the range of about 0.1 mg to about 3000 mg; wherein the unit dose is in the range of about 0.1 mg to about 1000 mg; wherein the unit dose is in the range of about 0.1 mg to about 500 mg; wherein the unit dose is in the range of about 0.1 mg to about 1000 mg; wherein the unit dose is in the range of about 0.1 mg to about 500 mg; wherein the unit dose is in the range of about 0.1 mg to about 100 mg; wherein the unit dose is in the range of about 1 mg to about 50 mg; wherein the unit dose is in the range of about 0.1 mg to about 10 mg; wherein the unit dose is in the range of about 0.5 mg to about 10 mg; wherein the unit dose is in the range of about 0.6 mg to about 6 mg; wherein the unit dose is about 0.1 mg; wherein the unit dose is about 0.2 mg; wherein the unit dose is about 0.3 mg; wherein the unit dose is about 0.4 mg; wherein the unit dose is about 0.5 mg: wherein the unit dose is about 0.6 mg; wherein the unit dose is about 0.7 mg; wherein the unit dose is about 0.8 mg; wherein the unit dose is about 0.9 mg; wherein the unit dose is about 1 mg; wherein the unit dose is about 2 mg; wherein the unit dose is about 3 mg; wherein the unit dose is about 4 mg; wherein the unit dose is about 5 mg; wherein the unit dose is about the range of 5 mg to about 10 mg; wherein the unit dose is about 6 mg; wherein the unit dose is about 7 mg; wherein the unit dose is about 8 mg; wherein the unit dose is about 9 mg; wherein the unit dose is about 10 mg; wherein the unit dose is about the range of 10 mg to about 15 mg; wherein the unit dose is about 11 mg; wherein the unit dose is about 12 mg; wherein the unit dose is about 13 mg; wherein the unit dose is about 14 mg; wherein the unit dose is about 15 mg; wherein the unit dose is about the range of 15 mg to about 20 mg; wherein the unit dose is about 16 mg; wherein the unit dose is about 17 mg; wherein the unit dose is about 18 mg; wherein the unit dose is about 19 mg; wherein the unit dose is about 20 mg; wherein the unit dose is about the range of 20 mg to about 30 mg; wherein the unit dose is about 25 mg; wherein the unit dose is about 30 mg; wherein the unit dose is about the range of 30 mg to about 40 mg; wherein the unit dose is about 35 mg; wherein the unit dose is about 40 mg; wherein the unit dose is about the range of 40 mg to about 50 mg; wherein the unit dose is about 45 mg; wherein the unit dose is about 50 mg; wherein the unit dose is about the range of 50 mg to about 100 mg; wherein the unit dose is about 75 mg; wherein the unit dose is about 100 mg; wherein the unit dose is about the range of 100 mg to about 200 mg; wherein the unit dose is about 150 mg; wherein the unit dose is about 200 mg; wherein the unit dose is about the range of 200 mg to about 300 mg; wherein the unit dose is about 200 mg; wherein the unit dose is about 250 mg; wherein the unit dose is about 300 mg; wherein the unit dose is about 350 mg; wherein the unit dose is about 400 mg; wherein the unit dose is about 450 mg; wherein the unit dose is about 500 mg; wherein the unit dose is about 750 mg; wherein the unit dose is about 1000 mg; wherein the unit dose is about 1500 mg; wherein the unit dose is about 2000 mg; wherein the unit dose is about 2500 mg; or wherein the unit dose is about 3000 mg.

In some embodiments of the foregoing embodiments of the composition for treatment of a disease, the unit dose is an immediate release formation. In some embodiments of the foregoing embodiments of the composition for treatment of a disease, the unit dose is an extended release formation. In some embodiments of the foregoing embodiments of the composition for treatment of a disease, the unit dose is a sustained release formation. In some embodiments of the foregoing embodiments of the composition for treatment of a disease, the unit dose is a controlled release formation. In some embodiments of the foregoing embodiments of the composition for treatment of a disease, the unit dose is an oral dosage form. In some embodiments, the oral dosage form is a tablet. In some embodiments of the foregoing embodiments of the composition for treatment of a disease, the oral dosage form is a capsule. In some embodiments of the foregoing embodiments of the composition for treatment of a disease, the unit dose is a capsule with no filler. In some embodiments of the foregoing embodiments of the composition for treatment of a disease, the oral dosage form is rapidly dissolving. In each of the foregoing embodiments, the disease may be independently selected from cancer and/or pain and/or pain related conditions. Non-limiting examples of cancer include breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. Non-limiting examples of pain and/or a pain related conditions include pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition. In some embodiments, the pain and/or pain related condition is selected from acute pain, chronic pain, neuropathic pain, nociceptive pain, and radicular pain.

In one aspect, the disclosure provides a method of treating cancer. In some embodiments, the method comprises administering to a patient in need thereof a therapeutic amount of dinitrophenol (DNP) or a pharmaceutically acceptable salt thereof, wherein the therapeutic amount of DNP is in the range of about 0.01 mg/kg of body weight to about 20 mg/kg of body weight. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer. In some embodiments, the DNP is administered in combination with an additional therapeutic agent. In some embodiments, the additional therapeutic agent comprises chemotherapy. In some embodiments, the additional therapeutic agent is selected from abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dalteparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, paclitaxel, pegaspargase, pegfilgrastim, peginterferon alpha-2b, pemetrexed, pentostatin, pipobroman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the additional therapeutic agent is doxorubicin or an analogue thereof. In some embodiments, the additional therapeutic agent is fulvestrant or an analogue thereof. In some embodiments, the additional therapeutic agent is paclitaxel or an analogue thereof. In some embodiments, the additional therapeutic agent is docetaxel or an analogue thereof. In some embodiments, the DNP is administered without chemotherapy. In some embodiments, the DNP is 2,4-DNP.

In some embodiments, the additional therapeutic agent comprises radiation therapy. Non-limiting examples of radiation therapy include high-energy radiation, e.g., x-rays, gamma rays, electron beams, or protons. In some embodiments, radiation therapy is useful to shrink tumors and destroy or damage cancer cells thus preventing them from growing and dividing.

In one aspect, the disclosure provides a method of treating pain and/or a pain related condition. In some embodiments, the method comprises administering to a patient in need thereof a therapeutic amount of dinitrophenol (DNP) or a pharmaceutically acceptable salt thereof, wherein the therapeutic amount of DNP is in the range of about 0.01 mg/kg of body weight to about 20 mg/kg of body weight. In some embodiments, the pain and/or pain related condition is selected from pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition. In some embodiments, the pain and/or pain related condition is selected from acute pain, chronic pain, neuropathic pain, nociceptive pain, and radicular pain. In some embodiments, the DNP is a prodrug of 2,4-DNP.

In some embodiments, the therapeutic amount of DNP is in the range of about 0.1 mg/kg of body weight to about 15 mg/kg of body weight, about 1 mg/kg of body weight to about 10 mg/kg of body weight, about 3 mg/kg of body weight to about 8 mg/kg of body weight, or about 4 mg/kg of body weight to about 6 mg/kg of body weight.

In some embodiments, the therapeutic amount of DNP is about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, or about 10 mg/kg of body weight. In some embodiments, the therapeutic amount of DNP is about 8 mg/kg of body weight.

In one aspect, the disclosure provides a method of treating cancer. In some embodiments, the method comprises administering to a patient in need thereof a therapeutic amount of a prodrug of dinitrophenol (DNP) or a pharmaceutically acceptable salt thereof, wherein the therapeutic amount of the prodrug of DNP is in the range of about 0.01 mg/kg of body weight to about 20 mg/kg of body weight. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer. In some embodiments, the DNP is administered in combination with chemotherapy. In some embodiments, the DNP is administered in combination with an additional therapeutic agent. In some embodiments, the additional therapeutic agent comprises chemotherapy. In some embodiments, the additional therapeutic agent is selected from abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dalteparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, paclitaxel, pegaspargase, pegfilgrastim, peginterferon alpha-2b, pemetrexed, pentostatin, pipobroman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the additional therapeutic agent is doxorubicin or an analogue thereof. In some embodiments, the additional therapeutic agent is fulvestrant or an analogue thereof. In some embodiments, the additional therapeutic agent is paclitaxel or an analogue thereof. In some embodiments, the additional therapeutic agent is docetaxel or an analogue thereof. In some embodiments, the DNP is administered without chemotherapy. In some embodiments, the additional therapeutic agent comprises radiation therapy. In some embodiments, the DNP is 2,4-DNP.

In one aspect, the disclosure provides a method of treating pain and/or a pain related condition. In some embodiments, the method comprises administering to a patient in need thereof a therapeutic amount of a prodrug of dinitrophenol (DNP) or a pharmaceutically acceptable salt thereof, wherein the therapeutic amount of the prodrug of DNP is in the range of about 0.01 mg/kg of body weight to about 20 mg/kg of body weight. In some embodiments, the pain and/or pain related condition is selected from pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition. In some embodiments, the pain and/or pain related condition is selected from acute pain, chronic pain, neuropathic pain, nociceptive pain, and radicular pain. In some embodiments, the DNP is a prodrug of 2,4-DNP.

In some embodiments, the therapeutic amount of the prodrug of DNP is in the range of about 0.001 mg/kg of body weight to about 15 mg/kg of body weight, about 0.01 mg/kg of body weight to about 15 mg/kg of body weight, about 0.001 mg/kg of body weight to about 100 mg/kg of body weight, about 0.01 mg/kg of body weight to about 100 mg/kg of body weight, about 0.1 mg/kg of body weight to about 15 mg/kg of body weight, about 0.001 mg/kg of body weight to about 0.1 mg/kg of body weight, about 0.001 mg/kg of body weight to about 0.01 mg/kg of body weight, about 0.01 mg/kg of body weight to about 0.1 mg/kg of body weight, about 0.1 mg/kg of body weight to about 100 mg/kg of body weight, about 1 mg/kg of body weight to about 100 mg/kg of body weight, about 1 mg/kg of body weight to about 20 mg/kg of body weight, about 5 mg/kg of body weight to about 10 mg/kg of body weight, about 7 mg/kg of body weight to about 9 mg/kg of body weight, about 10 mg/kg of body weight to about 20 mg/kg of body weight, about 15 mg/kg of body weight to about 17 mg/kg of body weight, about 50 mg/kg of body weight to about 100 mg/kg of body weight, about 60 mg/kg of body weight to about 90 mg/kg of body weight, about 75 mg/kg of body weight to about 85 mg/kg of body weight, or about 79 mg/kg of body weight to about 81 mg/kg of body weight.

In some embodiments, the therapeutic amount of the prodrug of DNP is about 0.001 mg/kg of body weight, about 0.002 mg/kg of body weight, about 0.003 mg/kg of body weight, about 0.004 mg/kg of body weight, about 0.005 mg/kg of body weight, about 0.006 mg/kg of body weight, about 0.007 mg/kg of body weight, about 0.008 mg/kg of body weight, about 0.009 mg/kg of body weight, about 0.01 mg/kg of body weight, about 0.02 mg/kg of body weight, about 0.03 mg/kg of body weight, about 0.04 mg/kg of body weight, about 0.05 mg/kg of body weight, about 0.06 mg/kg of body weight, about 0.07 mg/kg of body weight, about 0.08 mg/kg of body weight, about 0.09 mg/kg of body weight, about 0.1 mg/kg of body weight, about 0.2 mg/kg of body weight, about 0.3 mg/kg of body weight, about 0.4 mg/kg of body weight, about 0.5 mg/kg of body weight, about 0.6 mg/kg of body weight, about 0.7 mg/kg of body weight, about 0.8 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, or about 20 mg/kg of body weight. In some embodiments, the therapeutic amount of the prodrug of DNP is about 8 mg/kg of body weight. In some embodiments, the therapeutic amount of the prodrug of DNP is about 16 mg/kg of body weight.

In some embodiments, the therapeutic amount of the prodrug of DNP is about 71 mg/kg of body weight, about 72 mg/kg of body weight, about 73 mg/kg of body weight, about 74 mg/kg of body weight, about 75 mg/kg of body weight, about 76 mg/kg of body weight, about 77 mg/kg of body weight, about 78 mg/kg of body weight, about 79 mg/kg of body weight, 80 mg/kg of body weight, about 81 mg/kg of body weight, about 82 mg/kg of body weight, about 83 mg/kg of body weight, about 84 mg/kg of body weight, about 85 mg/kg of body weight, about 86 mg/kg of body weight, about 87 mg/kg of body weight, about 88 mg/kg of body weight, about 89 mg/kg of body weight, or about 90 mg/kg of body weight. In some embodiments, the therapeutic amount of the prodrug of DNP is about 80 mg/kg of body weight.

In some embodiments, the DNP is selected from 2,3-DNP, 2,4-DNP, 2,5-DNP, 2,6-DNP, 3,4-DNP, or 3,5-DNP. In some embodiments, the DNP is 2,4-DNP.

In some embodiments, the prodrug of DNP is selected from:
an amino acid (AA) ester of 2,3-DNP, an amino acid (AA) ester of 2,4-DNP, an amino acid (AA) ester of 2,5-DNP, an amino acid (AA) ester of 2,6-DNP, an amino acid (AA) ester of 3,4-DNP, or an amino acid (AA) ester of 3,5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10);
AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13);
amino carbamate 2,3-DNP prodrugs, amino carbamate 2,4-DNP prodrugs, amino carbamate 2,5-DNP prodrugs, amino carbamate 2,6-DNP prodrugs, amino carbamate 3,4-DNP prodrugs, or amino carbamate 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17);
amino carbonate 2,3-DNP prodrugs, amino carbonate 2,4-DNP prodrugs, amino carbonate 2,5-DNP prodrugs, amino carbonate 2,6-DNP prodrugs, amino carbonate 3,4-DNP prodrugs, or amino carbonate 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19);
phosphate analogs I-20, I-21, II-20, II-21, III-20, III-21, IV-20, IV-21, V-20, V-21, VI-20, and VI-21 (Schemes 1-6);
1,3 diketo analogs I-22 to I-32; II-22 to II-32; III-22 to III-32; IV-22 to IV-32; V-22 to V-32; and VI-22 to VI-32 (Schemes 1-6);
carbonate and carbamate analogs I-33 to I-39; II-33 to II-39; III-33 to III-39; IV-33 to IV-39; V-33 to V-39; and VI-33 to VI-39 (Schemes 1-6); and
benzoate analogs I-40, II-40, III-40, IV-40, V-40, and VI-40 (Schemes 1-6);
and combinations thereof.

In some embodiments, the prodrug of DNP is selected from:

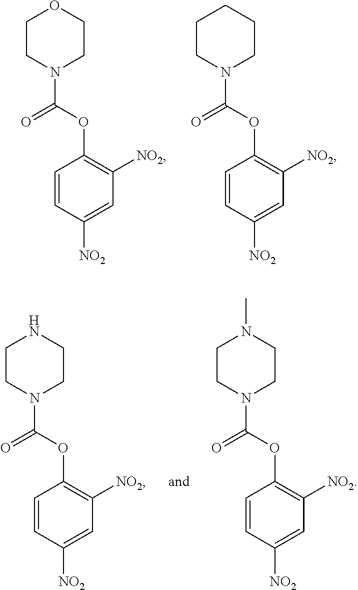

In some embodiments, the prodrug of DNP is

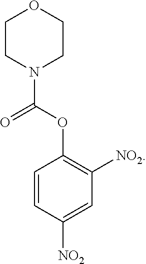 [SMILES: O=C(Oc1ccc([N+](=O)[O-])cc1[N+](=O)[O-])N1CCOCC1]

In some embodiments, the DNP and/or the prodrug of DNP is administered orally, intravenously, subcutaneously, or transdermally.

In some embodiments, the DNP and/or the prodrug of DNP is administered at a frequency of once a day, twice a day, three times a day, four times a day, once every two days, once every three days, once every four days, once every five days, once every six days, once a week, once every two weeks, once every three weeks, once a month. In some embodiments, the DNP and/or the prodrug of DNP is administered at a frequency of once a day. In some embodiments, the DNP and/or the prodrug of DNP is administered at a frequency of once a day for one day, once a day for two consecutive days, once a day for three consecutive days, once a day for four consecutive days, once a day for five consecutive days, once a day for six consecutive days, once a day for 7 consecutive days, or once a day for two consecutive weeks.

In one embodiment, the disclosure provides for a method of treatment of cancer using embodiments of the compositions and dosages described herein. In one embodiment, the disclosure relates to a method of treatment of breast cancer (including pre- and post-menopausal breast cancer) using embodiments of the compositions and dosages described herein. In one embodiment, the disclosure relates to a method of treatment of blood cancer using embodiments of the compositions and dosages described herein. In one embodiment, the disclosure relates to a method of treatment of cancer of the lymphatic system using embodiments of the compositions and dosages described herein. In one embodiment, the disclosure provides for a method of treatment of bone cancer using embodiments of the compositions and dosages described herein. In one embodiment, the disclosure relates to a method of treatment of brain cancer using embodiments of the compositions and dosages described herein. In one embodiment, the disclosure relates to a method of treatment of cervical cancer using embodiments of the compositions and dosages described herein. In one embodiment, the disclosure relates to a method of treatment of childhood cancer using embodiments of the compositions and dosages described herein. In one embodiment, the disclosure relates to a method of treatment of colorectal cancer using embodiments of the compositions and dosages described herein. In one embodiment, the disclosure relates to a method of treatment of head and/or neck cancer using embodiments of the compositions and dosages described herein. In one embodiment, the disclosure relates to a method of treatment of stomach cancer using embodiments of the compositions and dosages described herein. In one embodiment, the disclosure relates to a method of treatment of esophageal cancer using embodiments of the compositions and dosages described herein. In one embodiment, the disclosure relates to a method of treatment of kidney cancer using embodiments of the compositions and dosages described herein. In one embodiment, the disclosure relates to a method of treatment of liver cancer using embodiments of the compositions and dosages described herein. In one embodiment, the disclosure relates to a method of treatment of lung cancer using embodiments of the compositions and dosages described herein. In one embodiment, the disclosure relates to a method of treatment of metastatic cancer using embodiments of the compositions and dosages described herein. In one embodiment, the disclosure relates to a method of treatment of ovarian cancer using embodiments of the compositions and dosages described herein. In one embodiment, the disclosure relates to a method of treatment of pancreatic cancer using embodiments of the compositions and dosages described herein. In one embodiment, the disclosure relates to a method of treatment of prostate cancer using embodiments of the compositions and dosages described herein. In one embodiment, the disclosure relates to a method of treatment of sarcoma using embodiments of the compositions and dosages described herein. In one embodiment, the disclosure relates to a method of treatment of skin cancer using embodiments of the compositions and dosages described herein. In one embodiment, the disclosure relates to a method of treatment of testicular cancer using embodiments of the compositions and dosages described herein. In one embodiment, the disclosure relates to a method of treatment of thyroid cancer using embodiments of the compositions and dosages described herein. In one embodiment, the disclosure relates to a method of treatment of uterine cancer using embodiments of the compositions and dosages described herein.

In one embodiment, the disclosure relates to a method of treatment of pain and/or pain related condition using embodiments of the compositions and dosages described herein. In one embodiment, the disclosure relates to a method of treatment of acute pain using embodiments of the compositions and dosages described herein. In one embodiment, the disclosure relates to a method of treatment of chronic pain using embodiments of the compositions and dosages described herein. In one embodiment, the disclosure relates to a method of treatment of neuropathic pain using embodiments of the compositions and dosages described herein. In one embodiment, the disclosure relates to a method of treatment of nociceptive pain using embodiments of the compositions and dosages described herein. In one embodiment, the disclosure relates to a method of treatment of radicular pain using embodiments of the compositions and dosages described herein. In one embodiment, the disclosure relates to a method of treatment of fibromyalgia using embodiments of the compositions and dosages described herein.

In one embodiment, the disclosure relates to a method of treatment of cancer and/or pain and/or pain related conditions using embodiments of the compositions and dosages described herein. In one such embodiment, such dosages for an adult independently ranging from: 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day; about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; about 10 mg/day to about 150 mg/day; about 20 mg/day to about 150 mg/day; about 30 mg/day to about 150 mg/day; about 40 mg/day to about 150 mg/day; about 50 mg/day to about 150 mg/day; about 60 mg/day to about 150 mg/day; about 70 mg/day to about 150 mg/day; about 80 mg/day to about 150 mg/day; about 90 mg/day to about 150 mg/day; about 80 mg/day to about 100 mg/day; about 90 mg/day to about 100 mg/day; about 91 mg/day to about 100 mg/day 92 mg/day to about 100 mg/day; about 93 mg/day to about 100 mg/day; about 94 mg/day to about 100 mg/day; or independently dosages of about 90 mg/day; about 91 mg/day; about 92 mg/day; about 93 mg/day; about 94 mg/day; about 95 mg/day; about 96 mg/day; about 97 mg/day; about 98 mg/day or about 99 mg/day. In one such embodiment, such dosages for an adolescent independently ranging from: about 1 mg/day to about 45 mg/day; about 1 mg/day to about 50 mg/day; about 5 mg/day to about 45 mg/day; about 5 mg/day to about 50 mg/day; about 10 mg/day to about 45 mg/day; about 15 mg/day to about 45 mg/day; about 20 mg/day to about 45 mg/day; about 25 mg/day to about 45 mg/day; about 30 mg/day to about 45 mg/day; about 35 mg/day to about 45 mg/day; about 35 mg/day to about 40 mg/day or; independently dosages of about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.1 mg/day, about 1.2 mg/day, about 1.3 mg/day, about 1.4 mg/day, about 1.5 mg/day, about 1.6 mg/day, about 1.7 mg/day, about 1.8 mg/day, about 1.9 mg/day, about 2 mg/day, about 2.1 mg/day, about 2.2 mg/day, about 2.3 mg/day, about 2.4 mg/day, about 2.5 mg/day, about 2.6 mg/day, about 2.7 mg/day, about 2.8 mg/day, about 2.9 mg/day, about 3 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day, about 8 mg/day, about 8.1 mg/day, about 8.2 mg/day, about 8.3 mg/day, about 8.4 mg/day, about 8.5 mg/day, about 8.6 mg/day, about 8.7 mg/day, about 8.8 mg/day, about 8.9 mg/day, about 9 mg/day, about 9.1 mg/day, about 9.2 mg/day, about 9.3 mg/day, about 9.4 mg/day, about 9.5 mg/day, about 9.6 mg/day, about 9.7 mg/day, about 9.8 mg/day, about 9.9 mg/day, or about 10 mg/day about 35 mg/day; about 36 mg/day; about 37 mg/day; about 38 mg/day; about 39 mg/day; about 40 mg/day; about 41 mg/day; about 42 mg/day; about 43 mg/day; about 44 mg/day or about 45 mg/day. In another such embodiment, such dosages for an adult independently ranging from: 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day; about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; about 10 mg/day to about 150 mg/day; about 20 mg/day to about 150 mg/day; about 30 mg/day to about 150 mg/day; about 40 mg/day to about 150 mg/day; about 50 mg/day to about 150 mg/day; about 60 mg/day to about 150 mg/day; about 70 mg/day to about 150 mg/day; about 80 mg/day to about 150 mg/day; about 90 mg/day to about 150 mg/day; about 80 mg/day to about 100 mg/day; about 90 mg/day to about 100 mg/day; about 91 mg/day to about 100 mg/day; about 92 mg/day to about 100 mg/day; about 93 mg/day to about 100 mg/day: about 94 mg/day to about 100 mg/day; or independently dosages of about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.1 mg/day, about 1.2 mg/day, about 1.3 mg/day, about 1.4 mg/day, about 1.5 mg/day, about 1.6 mg/day, about 1.7 mg/day, about 1.8 mg/day, about 1.9 mg/day, about 2 mg/day, about 2.1 mg/day, about 2.2 mg/day, about 2.3 mg/day, about 2.4 mg/day, about 2.5 mg/day, about 2.6 mg/day, about 2.7 mg/day, about 2.8 mg/day, about 2.9 mg/day, about 3 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day, about 8 mg/day, about 8.1 mg/day, about 8.2 mg/day, about 8.3 mg/day, about 8.4 mg/day, about 8.5 mg/day, about 8.6 mg/day, about 8.7 mg/day, about 8.8 mg/day, about 8.9 mg/day, about 9 mg/day, about 9.1 mg/day, about 9.2 mg/day, about 9.3 mg/day, about 9.4 mg/day, about 9.5 mg/day, about 9.6 mg/day, about 9.7 mg/day, about 9.8 mg/day, about 9.9 mg/day, or about 10 mg/day; about 90 mg/day; about 91 mg/day; about 92 mg/day; about 93 mg/day; about 94 mg/day; about 95 mg/day; about 96 mg/day; about 97 mg/day; about 98 mg/day or about 99 mg/day. In another such embodiment, such dosages for an adolescent independently ranging from: 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day; about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; about 1 mg/day to about 45 mg/day; about 1 mg/day to about 50 mg/day; about 5 mg/day to about 45 mg/day; about 5 mg/day to about 50 mg/day; about 10 mg/day to about 45 mg/day; about 15 mg/day to about 45 mg/day; about 20 mg/day to about 45 mg/day; about 25 mg/day to about 45 mg/day; about 30 mg/day to about 45 mg/day; about 35 mg/day to about 45 mg/day; about 35 mg/day to about 40 mg/day or; independently dosages of about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.1 mg/day, about 1.2 mg/day, about 1.3 mg/day, about 1.4 mg/day, about 1.5 mg/day, about 1.6 mg/day, about 1.7 mg/day, about 1.8 mg/day, about 1.9 mg/day, about 2 mg/day, about 2.1 mg/day, about 2.2 mg/day, about 2.3 mg/day, about 2.4 mg/day, about 2.5 mg/day, about 2.6 mg/day, about 2.7 mg/day, about 2.8 mg/day, about 2.9 mg/day, about 3 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day, about 8 mg/day, about 8.1 mg/day, about 8.2 mg/day, about 8.3 mg/day, about 8.4 mg/day, about 8.5 mg/day, about 8.6 mg/day, about 8.7 mg/day, about 8.8 mg/day, about 8.9 mg/day, about 9 mg/day, about 9.1 mg/day, about 9.2 mg/day, about 9.3 mg/day, about 9.4 mg/day, about 9.5 mg/day, about 9.6 mg/day, about 9.7 mg/day, about 9.8 mg/day, about 9.9 mg/day, or about 10 mg/day; about 35 mg/day; about 35 mg/day; about 37 mg/day; about 38 mg/day; about 39 mg/day; about 40 mg/day; about 41 mg/day; about 42 mg/day; about 43 mg/day; about 44 mg/day or about 45 mg/day. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer. In some embodiments, the DNP is administered in combination with chemotherapy. In some embodiments, the DNP is administered in combination with an additional therapeutic agent. In some embodiments, the additional therapeutic agent comprises chemotherapy. In some embodiments, the additional therapeutic agent is selected from abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dalteparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, paclitaxel, pegaspargase, pegfilgrastim, peginterferon alpha-2b, pemetrexed, pentostatin, pipobroman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof. In some embodiments, the additional therapeutic agent is doxorubicin or an analogue thereof. In some embodiments, the additional therapeutic agent is fulvestrant or an analogue thereof. In some embodiments, the additional therapeutic agent is paclitaxel or an analogue thereof. In some embodiments, the additional therapeutic agent is docetaxel or an analogue thereof. In some embodiments, the DNP is administered without chemotherapy. In some embodiments, the additional therapeutic agent comprises radiation therapy. In some embodiments, the pain and/or pain related condition is selected from pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition. In some embodiments, the pain and/or pain related condition is selected from acute pain, chronic pain, neuropathic pain, nociceptive pain, and radicular pain.

In one embodiment, the disclosure relates to a method of treatment of cancer and/or pain and/or pain related conditions using embodiments of the compositions and dosages described herein. In one such embodiment, such dosages independently ranging from: 0.1 mg/day to about 50 mg/day; 0.1 mg/day to about 50 mg/day; about 0.1 mg/day to about 45 mg/day; about 0.1 mg/day to about 40 mg/day; about 0.1 mg/day to about 35 mg/day; about 0.1 mg/day to about 30 mg/day; about 0.1 mg/day to about 25 mg/day, about 0.1 mg/day to about 20 mg/day; about 0.1 mg/day to about 15 mg/day; about 0.1 mg/day to about 10 mg/day; about 0.1 mg/day to about 6 mg/day; about 0.1 mg/day to about 5 mg/day; about 0.1 mg/day to about 4 mg/day; about 0.1 mg/day to about 3 mg/day; about 0.1 mg/day to about 2 mg/day; about 0.6 mg/day to about 10 mg/day; about 0.6 mg/day to about 6 mg/day; 1 mg/day to about 45 mg/day; about 5 mg/day to about 45 mg/day; about 10 mg/day to about 45 mg/day; about 15 mg/day to about 45 mg/day; about 20 mg/day to about 45 mg/day; about 25 mg/day to about 45 mg/day; about 30 mg/day to about 45 mg/day: about 35 mg/day to about 45 mg/day; about 35 mg/day to about 40 mg/day or; about independently dosages of 31 mg/day; about 32 mg/day; about 33 mg/day; about 34 mg/day; about 35 mg/day; about 36 mg/day; about 37 mg/day; about 38 mg/day; about 39 mg/day or 40 mg/day. In another such embodiment, such dosages independently ranging from: about 1 mg/day to about 45 mg/day; about 5 mg/day to about 45 mg/day; about 10 mg/day to about 45 mg/day; about 15 mg/day to about 45 mg/day; about 20 mg/day to about 45 mg/day; about 25 mg/day to about 45 mg/day; about 30 mg/day to about 45 mg/day; about 35 mg/day to about 45 mg/day; about 35 mg/day to about 40 mg/day or; independently dosages of about 0.1 mg/day, about 0.2 mg/day, about 0.3 mg/day, about 0.4 mg/day, about 0.5 mg/day, about 0.6 mg/day, about 0.7 mg/day, about 0.8 mg/day, about 0.9 mg/day, about 1 mg/day, about 1.1 mg/day, about 1.2 mg/day, about 1.3 mg/day, about 1.4 mg/day, about 1.5 mg/day, about 1.6 mg/day, about 1.7 mg/day, about 1.8 mg/day, about 1.9 mg/day, about 2 mg/day, about 2.1 mg/day, about 2.2 mg/day, about 2.3 mg/day, about 2.4 mg/day, about 2.5 mg/day, about 2.6 mg/day, about 2.7 mg/day, about 2.8 mg/day, about 2.9 mg/day, about 3 mg/day, about 3.1 mg/day, about 3.2 mg/day, about 3.3 mg/day, about 3.4 mg/day, about 3.5 mg/day, about 3.6 mg/day, about 3.7 mg/day, about 3.8 mg/day, about 3.9 mg/day, about 4 mg/day, about 4.1 mg/day, about 4.2 mg/day, about 4.3 mg/day, about 4.4 mg/day, about 4.5 mg/day, about 4.6 mg/day, about 4.7 mg/day, about 4.8 mg/day, about 4.9 mg/day, about 5 mg/day, about 5.1 mg/day, about 5.2 mg/day, about 5.3 mg/day, about 5.4 mg/day, about 5.5 mg/day, about 5.6 mg/day, about 5.7 mg/day, about 5.8 mg/day, about 5.9 mg/day, about 6 mg/day, about 6.1 mg/day, about 6.2 mg/day, about 6.3 mg/day, about 6.4 mg/day, about 6.5 mg/day, about 6.6 mg/day, about 6.7 mg/day, about 6.8 mg/day, about 6.9 mg/day, about 7 mg/day, about 7.1 mg/day, about 7.2 mg/day, about 7.3 mg/day, about 7.4 mg/day, about 7.5 mg/day, about 7.6 mg/day, about 7.7 mg/day, about 7.8 mg/day, about 7.9 mg/day, about 8 mg/day, about 8.1 mg/day, about 8.2 mg/day, about 8.3 mg/day, about 8.4 mg/day, about 8.5 mg/day, about 8.6 mg/day, about 8.7 mg/day, about 8.8 mg/day, about 8.9 mg/day, about 9 mg/day, about 9.1 mg/day, about 9.2 mg/day, about 9.3 mg/day, about 9.4 mg/day, about 9.5 mg/day, about 9.6 mg/day, about 9.7 mg/day, about 9.8 mg/day, about 9.9 mg/day, or about 10 mg/day; about 31 mg/day; about 32 mg/day; about 33 mg/day; about 34 mg/day; about 35 mg/day; about 36 mg/day; about 37 mg/day; about 38 mg/day; about 39 mg/day or about 40 mg/day. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, and uterine cancer. In some embodiments, the DNP is administered in combination with chemotherapy. In some embodiments, the DNP is administered without chemotherapy. In some embodiments, the additional therapeutic agent comprises radiation therapy. In some embodiments, the pain and/or pain related condition is selected from pain associated with a medical condition selected from neuropathic pain, osteoarthritis, rheumatoid arthritis, fibromyalgia, and back, musculoskeletal pain, Ankylosing spondylitis, juvenile rheumatoid arthritis, migraines, dental pain, abdominal pain, ischemic pain, postoperative pain, and pain due to an anesthetic or surgical condition. In some embodiments, the pain and/or pain related condition is selected from acute pain, chronic pain, neuropathic pain, nociceptive pain, and radicular pain.

In some embodiments, the unit dose is delivered intravenously. In some embodiments, the unit dose is delivered by means of an intravenous drip along with saline. In some embodiments, the unit dose is delivered by means of an intravenous drip along with saline, other medications, vitamins and/or nourishment. In some embodiments, the unit dose is delivered subcutaneously. In some embodiments, the unit dose is delivered topically. In some embodiments, the unit dose is delivered transdermally. In some embodiments, the unit dose is in the form of a patch.

The dose may be administered as a single daily dose, a twice-daily dose, three times daily, or more frequently. The dose may be administered three times weekly, twice weekly, once weekly, or less frequently. In an embodiment, administration frequency may be between 1 and 5 times a day. In another embodiment, administration frequency may be between 2 and 4 times a day. In another embodiment, administration frequency may be at least 3 times a day. In another embodiment, administration frequency may be twice a day. In another embodiment, administration frequency may be once a day. In another embodiment, administration frequency may be less frequent than once a day. In other embodiments, administration frequency may be once every 2 days or once every 3 days or once every 4 days or once every 5 days or once every 6 days. In another embodiment, administration frequency may be once a week. In another embodiment, administration frequency may change with time, starting at a certain rate, such as once or twice a day, and then decreasing to less frequently, such as once every 2 days or once every 3 days, or once a week, after the first day of treatment. In another embodiment, administration frequency may change with time, starting at a certain rate, such as once or twice a day, and then decreasing to less frequently, such as once every 2 days or once every 3 days, or once a week, after the first two or three days of treatment. In another embodiment, administration frequency may change with time, starting at a certain rate, such as once or twice a day, and then decreasing to less frequently, such as once every 2 days or once every 3 days, or once a week, after the first week of treatment. In another embodiment, administration frequency may be on demand, as therapeutic treatment is required or desired.

It will be understood, based on the disclosure encompassed herein, how to determine whether a subject needs an additional and/or continued dose. It will also be understood that the selected dosing frequency may require an adjustment of the dosage of active ingredient. It will also be understood, based on the disclosure encompassed herein, that the selected dosage of active ingredient may require an adjustment of the dosing frequency. The disclosure encompassed herein, in combination with the skill in the art, will enable the skilled artisan to optimize both the dosage of the active ingredient and the frequency of administration of the active ingredient to treat a subject in need thereof.

The unit dose may also be adjusted based upon the size of the patient. In one embodiment, the numbers provided herein are based upon a 60 kg patient. The same therapy could be provided for a smaller or larger sized patient, by respectively reducing or increasing the dose size. By way of example only, a 20 kg child patient would need a much smaller dose than a 60 kg adult patient.

Formulation approaches are employed such as controlled release technologies (polymers, liposomes, etc.) to achieve once a day PK profile for DNP. In some embodiments, DNP prodrugs and bioprecursors with linkers containing open functional groups are synthesized, which allows conjugation of each of these entities to nanoparticles, such as dendrimers, in order to modulate the pharmacokinetics of the molecule to enable “trickle” drug delivery. Such DNP prodrugs and bioprecursors are delivered as depot nanoparticle formulation that release DNP in a slow, sustained fashion at low doses, compared to dose and release of DNP alone, to avoid possible toxicity issues. The in vitro stability, in vivo plasma release kinetics and PK profiles are evaluated. In vivo studies are carried out in Sprague-Dawley rats. LC/MS/MS is used to analyze plasma DNP released from the various prodrug-nanoparticle formulations to determine the PK profile of DNP release in the rat model.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: DNP for the Treatment of Metastatic Breast Cancer

In view of data demonstrating that metastasis is largely driven by nuclear reactive oxygen species (ROS), this Example is related to developing DNP, a drug candidate that suppresses ROS production as a novel therapeutic for stage IV metastatic breast cancer. DNP is already in clinical trials for neurodegenerative diseases and preliminary evidence indicates it can reduce ROS production in mitochondria thereby suppressing their accumulation in the nucleus of breast cancer cells. It is hypothesized that mitochondrial dysfunction, a hallmark of advanced stage cancer, increases mitochondrial reactive oxygen species (ROS) production to drive chromatin structural changes (chromatin folding heterogeneity) responsible for the dysregulation of the expression of several oncogenes responsible for the establishment and progression of metastatic disease. A pilot study is performed to test the efficacy of DNP, a mitochondrial uncoupler, as an anti-metastatic agent and chemosensitizer to further existing breast cancer therapeutics, and can be expanded to other types of tumors (e.g., lung, pancreatic, prostate).

It is determined if: 1) if mitochondrial uncouplers reduce the levels of ROS in the cancer cell nucleus thereby suppressing chromatin folding heterogeneity linked to increased phenotypic plasticity and EMT and 2) test in mouse xenograft models whether the combination of DNP and chemotherapeutics yields better outcomes (lower tumor burden, reduced metastasis, prolongation of life) than chemotherapeutics alone. Then, these compounds are tested and applied to the clinical care of multiple types of tumors affecting the elderly, and new combination therapeutics are developed.

It is demonstrated that suppressing mitochondrial ROS production reduces chromatin folding heterogeneity and tumor cell plasticity, tumor evolution to deadly forms of cancer as well as in vivo testing to show whether combination therapies that include mitochondria uncouplers, already in clinical trials, provide better treatments compared to the standard of care alone.

Determine if DNP reduces ROS-induced chromatin folding and gene expression heterogeneity in breast cancer cells and if this suppresses clonogenicity and invasiveness. Whether DNP functions as a chromatin protective agent suppressing ROS-induced damage to chromatin is tested. Chromatin folding heterogeneity is directly measured using partial wave spectroscopy microscopy (PWS) combined with ChromSTEM. In addition, scRNAseq studies are developed using cell lines and patient-derived organoids to validate structural chromatin data obtained from PWS is examined. Also, whether DNP reduces the ability of tumor cells to colonize soft agar or invade Matrigel is examined using two established assays to examine in vitro metastatic potential.

Develop a pilot study in vivo comparing the effectiveness of combination therapies of doxorubicin and fulvestrant with DNP vs chemotherapy alone. Xenograft mouse models are used to determine if the inclusion of DNP as a component of treatment improves responsiveness of breast tumor xenografts to either doxorubicin or fulvestrant. Mice bearing the xenografts are treated with either compound alone or in combination with DNP. Tumor growth, tumor burden and metastasis is assessed.

This Example describes developing the central conceptual and translational pillars for the development of a completely novel therapeutic strategy for metastatic breast cancer affecting for example, women without a clear predisposing genetic driver. As demonstrated herein nuclear ROS are sufficient and required for metastatic cancer evolution towards deadly disease and DNP as a pharmacologic tool to inhibit this process. This Examples provides novel clinical trials of existing drug candidates for the treatment of metastatic breast cancer.

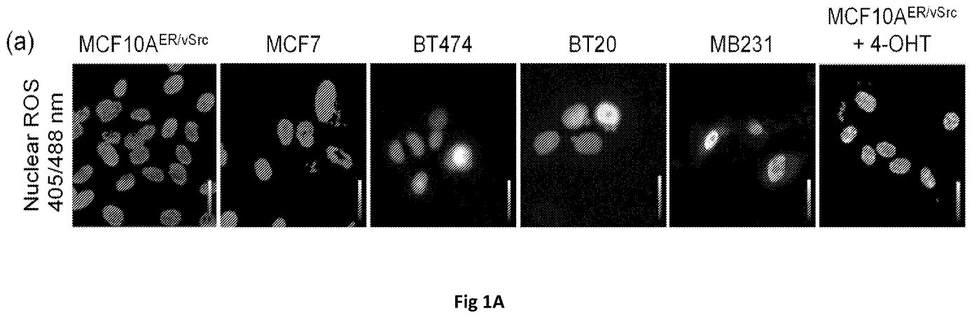

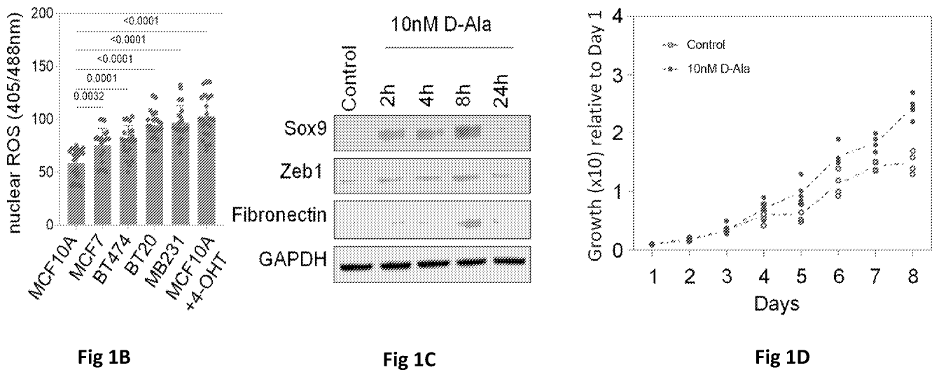

Having discovered this novel role of nuclear ROS in chromatin remodeling, it was tested that removing nuclear ROS enhances the curative effect of chemotherapy and could even drive metastasis into remission by suppressing the plasticity metastatic cells require to survive in microenvironments different than the organ of origin. Using a genetically encoded antioxidant NLS-catalase drove established metastatic disease into remission while bolstering the therapeutic effect of doxorubicin. These results indicate that (1) removing nuclear ROS is lethal for metastatic cancer cells; (2) removing nuclear ROS is safe for the host; and (3) combining nuclear ROS removal with traditional chemotherapy may be effective towards treating stage IV breast cancer that is currently incurable and highly lethal. Hence, it is proposed to repurpose DNP, a drug that efficiently suppresses mitochondria ROS production and, as shown in FIGS. 1A-1D, nuclear ROS accumulation as a novel adjuvant therapeutic for stage IV breast cancer.

There are major scientific and clinical roadblocks preventing the development of better therapeutics for stage IV breast cancer including: (1) lack of a better understanding of the molecular drivers of metastasis and (2) lack of lead drug candidates to target these drivers. The data described herein (e.g. FIGS. 1A-1D) provides supporting evidence including: (1) data indicating that mitochondrial H₂O₂ promotes EMT in cancer cells due to a newly discovered signaling function; (2) data showing that suppressing nuclear H₂O₂-induced chromatin folding heterogeneity significantly drives metastasis into remission and bolsters the effectiveness of traditional chemotherapy and (3) introduces a novel technology partial wave spectroscopy microscopy that allows to directly assess ROS-induce chromatin folding heterogeneity.

Nuclear ROS drive EMT, supports tumor growth and enable metastasis of breast cancer. Aggressive breast cancer cells have higher steady state ROS levels in the nucleus at baseline. Also, nuclear ROS drive their aggressive behavior both in vitro and in vivo and suppressing them with a genetically encoded antioxidant directed to the nucleus (NLS-catalase) both restores sensitivity to traditional chemotherapy (i.e. chemotherapy) and drives metastatic disease into remission.

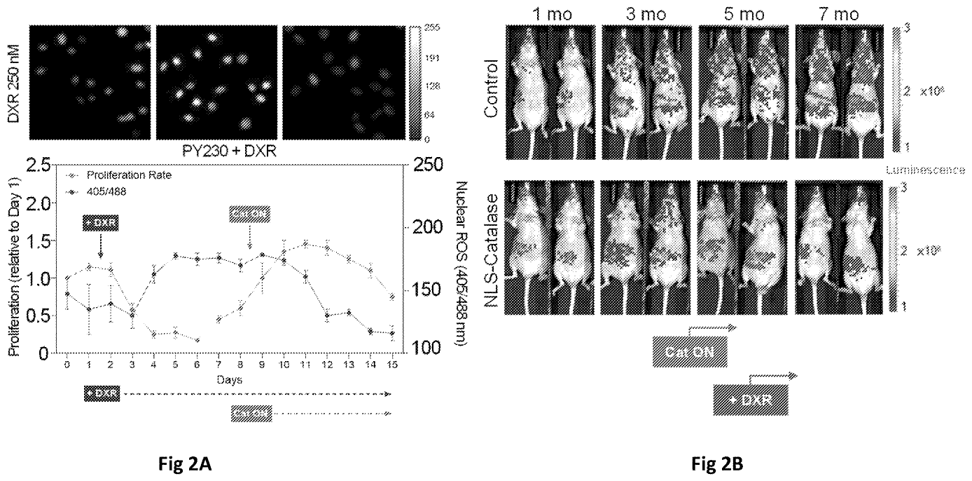

Nuclear ROS are sufficient and required to activate EMT and metastasis of breast cancer cells. ROS levels were measured in the nuclei of different breast cancer cell lines displaying vastly different tumorigenicity and metastatic potential using novel NLS-Orp1roGFP2 biosensors. Orp1roGFP2 is a fluorescence based biosensor that displays distinct excitation wavelengths for the reduced form (\=488 nm) and oxidized forms (λ=405 nm) with no spectral overlap and a midpoint oxidation potential of Eo=−230 mV ideally suited to capture subtle changes in ROS levels in most organelles (i.e. cytoplasm, −230 mV; peroxisome, −270 mV; nucleus, −280 mV and mitochondria, −330 mV). As shown in FIGS. 1A-1D, non-cancerous mammary epithelial MCF10A cells, representing healthy mammary cells, had the lowest levels of nuclear ROS (colored with blue tones) while the highly metastatic MDA-MB231 cell line showed the most nuclear ROS (pseudo-colored yellow/red). Also, transformation of MCF10A by v-Src promoted a marked increase in nuclear ROS levels (FIGS. 1A & B). These results show that aggressive cancer cells exist at a chronically high nuclear ROS state. To directly link nuclear ROS with aggressive cancer behavior, a nucleus-targeted system (NLS-D-amino acid oxidase, NLS-DAO) was engineered that allows to generate ROS21 directly in the nucleus and expressed it into MCF10A cells to recreate, using a non-transformed cell background, this distinctive feature of metastatic breast cancer cell lines. Results in FIG. 1C indicated that introducing ROS into the nucleus of MCF10A triggered EMT genes involved in malignant transformation as well as increased the rate of breast cancer cell proliferation (FIG. 1D). These results indicate that nuclear ROS drive EMT and promotes cancer cell plasticity associated with the emergence of more aggressive cancer cell phenotypes. Next, it was tested whether nuclear ROS contributed to treatment failure by promoting resistance to first line chemotherapeutic drugs, i.e. doxorubicin. It was also tested whether removing suppressing ROS specifically in the nucleus restored sensitivity to doxorubicin. The strategy of removing ROS from the nucleus is novel, targets a feature that seems unique to cancer cells and preserves redox signaling that happen in large part in the cytosol22. Results in FIG. 2A indicate that the application of doxorubicin (DXR) to murine mammary cancer cells PY230, eliminated the bulk of cells within a few hours from application (see blue line). However, persisting cells distinctively showed high levels of nuclear ROS (see purple line) and resumed proliferation in the presence of chemotherapy on day 6. Activation of NLS-catalase expression on day 9 suppressed nuclear ROS (purple line) while resensitizing therapy resistant cells to doxorubicin.

Results in FIG. 2A, closely recapitulate the reality of most clinical cancer treatments that are effective at first eliminating the bulk tumor but that are become ineffective when the disease recur. In addition, results place nuclear ROS at the core of resistance and their elimination as a novel strategy to enhance cancer therapeutics. To directly test this idea xenograft experiments were performed using female NOD/SCID immuno-compromised mice. Highly metastatic v-Src transformed luciferase-expressing MCF10A were implanted into the mammary fat pad of NOD/SCID mice and left to develop into metastatic disease. Three months after implantation, metastases were clearly detected by luciferin-induced bioluminescence in the chest and head of most mice FIG. 2B. Following the detection of distant metastases (stage IV), NLS-catalase knocked into the cells under a TetON-inducible promoter was induced by the addition of tetracycline into the chow. Expression of NLS-catalase for about 2 months drove established metastatic disease into full remission while suppressing the development of new lesions. Surprisingly, primary tumors were less affected indicating that nuclear ROS-induced cell plasticity is required for the survival of metastatic cells at sites vastly different than the tissue of origin but not primary tumors developing at their organ of origin. Therefore, the strategy to combine nuclear ROS elimination (with NLS-catalase) and traditional chemotherapy (doxorubicin) was tested. Results assessed at month 7 after 2 months of combined therapies indicated complete remission of metastatic disease as well as significant regression of primary tumor. While not wishing to be bound by any particular theory, these results suggest that combination therapies that target both nuclear ROS and the primary tumor may be effective for the treatment of stage IV breast cancer.

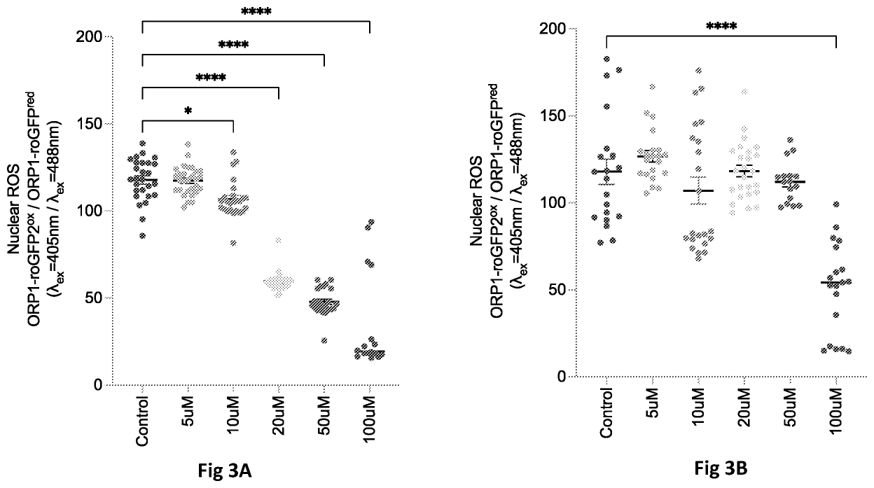

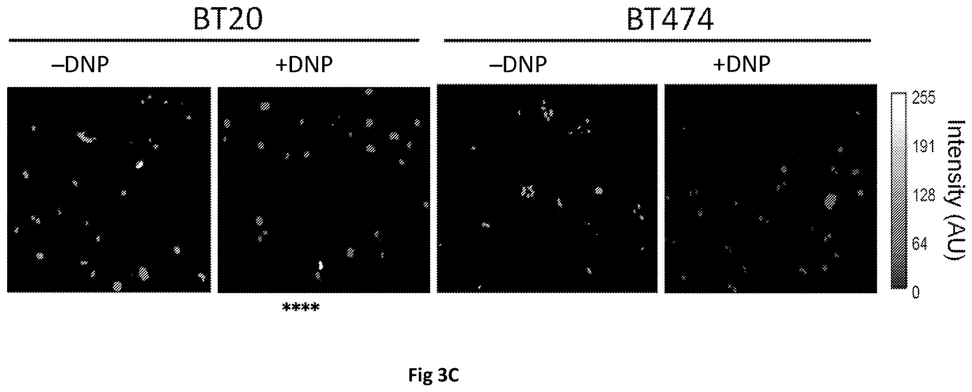

Currently, it is not possible to safely use genetic manipulation to treat human patients, highlighting the need to develop and test pharmacologic agents that recapitulate the activity of NLS-catalase in suppressing nuclear ROS. FIGS. 3A-3C shows evidence that DNP strongly suppresses the accumulation of nuclear ROS in two highly metastatic human breast cancer cell lines BT20 and BT474. It is proposed that DNP, a drug with well-established toxicologic as well as tolerability profiles already in clinical trials, can be developed as an adjuvant therapy for stage IV incurable breast cancer based at least on results shown in FIGS. 2A-2B.

Determine if DNP reduces ROS-induced chromatin folding and gene expression heterogeneity in breast cancer cells and if this suppresses clonogenicity and invasiveness. Having determined that nuclear ROS are centrally involved in chemotherapy failure and metastatic dissemination tumors that are driven by nuclear ROS and would respond to the proposed therapies are distinguished from those that would not. Data showing a suite of novel technologies that have been developed to directly assess chromatin folding heterogeneity, a major outcome of ROS activity in the nucleus that drives gene expression involved in tumor progression towards chemoresistance and incurable metastatic disease, are discussed below.

Imaging and Computational Platform to Study the Effects of Chromatin Topology on Transcription. It is determined if chromatin folding heterogenization induced by nuclear ROS can prognosticate chemoresistance and metastatic potential of breast cancer cells.

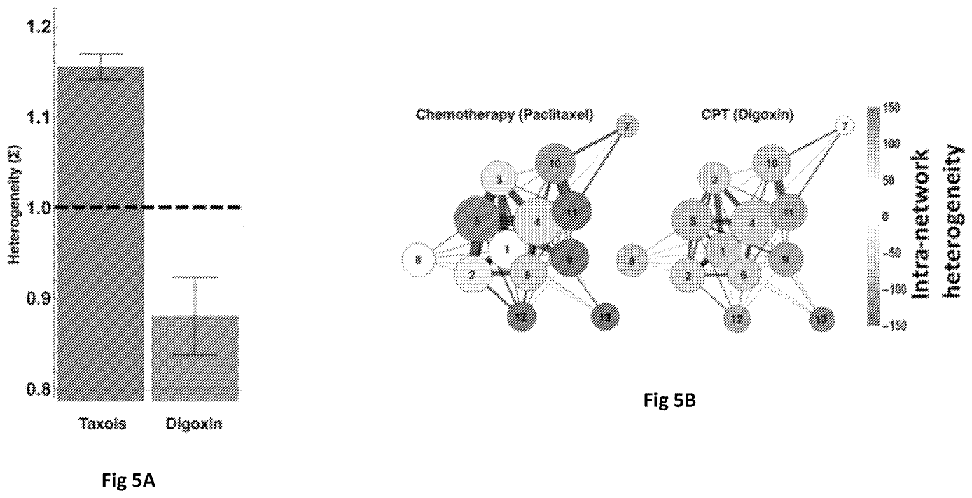

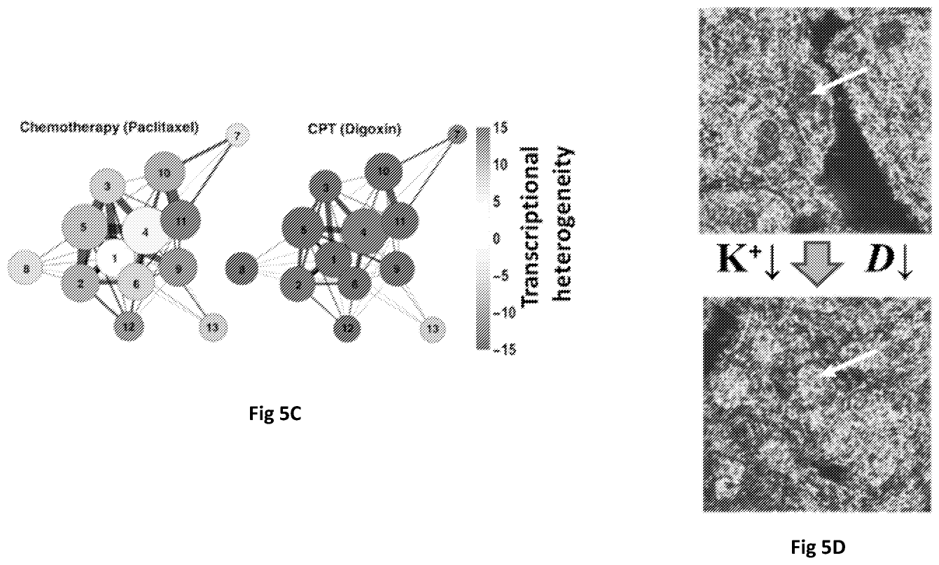

Chromatin Folding Heterogeneity Is a Critical Event in both Tumorigenesis and Cancer Evolution. Further, in cell culture, is has been found that chromatin heterogeneity is higher in treatment resistant variants of cancer cell lines and in cancer cells that evade chemotherapy (FIG. 5). Hence, PWS is used to test the hypothesis that nuclear ROS promote while DNP suppresses chromatin folding heterogenization, tumor chemoresistance and metastasis.

Determine if DNP reduces ROS-induced chromatin folding and gene expression heterogeneity in breast cancer cells and if this suppresses clonogenicity and invasiveness.

Cell lines already determined to display significant accumulation of nuclear ROS as well as invasive and metastatic potential (BT20, BT474, MDA-MB231) are used. Significant heterogeneity was found in steady state levels of ROS in the nucleus of these lines as denoted by the vastly different heat map intensities shown across different cells in the microscopy field (FIG. 1A). Having found that DNP suppresses the outflow of ROS into the nucleus (FIGS. 3A-3C) it is examined whether it also suppresses chromatin folding heterogeneity using PWS as shown in FIGS. 4 and 5A-5D. In addition, single cell RNA-seq is performed to determine if the reduction in nuclear ROS also reduces cell to cell transcriptomic diversification. Samples passing quality control are loaded onto 10× Genomics Chromium Cytometer for partitioning and encapsulation of single cells into nanoliter-sized GEMs (Gel beads-in-EMulsion). Each encapsulated cell is lysed within its GEM. The total RNA released is reverse transcribed to cDNA with primers attached to the gel bead, each of which carries a unique 10× barcode for downstream cell separation. Subsequently, the GEMs are broken, and all uniquely barcoded cDNAs are pooled and then PCR amplified to generate enough material for Illumina sequencing. Raw sequencing data, are demultiplexed using Cell Ranger from 10× Genomics, converting the raw data into FASTQ format. Cell Ranger are also used to align the FASTQ files to the appropriate reference genome and count the number of reads from each cell. This generates a matrix file summarizing alignment results that are imported for further analysis. Each individual sample is preprocessed, normalized and scaled. All samples are then combined into a single dataset, adding metadata with the original sample information. This combined dataset is evaluated for differences between samples and/or clusters using the array of tools available, including finding biomarkers (to help identify cell phenotypes) and performing differential expression comparisons between specific clusters. In addition, such phenotypes are characterized regarding their ability to form colonies when cells are grown under low adherence (i.e. on top of soft agar) as well as invade Matrigel™ a basal membrane mimetic. These parameters are generally taken as adequate proxies to assess the metastatic potential. Based on results, it is anticipated DNP will suppress transcriptomic heterogeneity directly linked to the ability of the cells to adapt to different media, acquire phenotypic plasticity and metastasize.

In a non-limiting example, cell line models recapitulate several major aspects of cancer affecting humans, subtle details that are critical for treatment success or failure are missed, and a select number of experiments using human organoids are repeated. Organoids are derived from patients samples and far better represent tumors evolving in humans.

Develop a pilot study in vivo comparing the effectiveness of combination therapies of doxorubicin and fulvestrant, DNP vs chemotherapy alone.

The hypothesis that mitigating mitochondrial dysfunction via scavenging ROS prevents chromatin changes that promote treatment resistance is tested using DNP, a compound in development with uncoupling activity already in Phase I trials for Huntington's Disease. DNP works largely by reducing the mitochondrial electrochemical potential reducing the output of ROS. Though DNP, like most drugs currently in use, can be toxic at high doses, produced safety data has been produced showing that DNP at the doses proposed is safe for inpatient administrative and effective for the purpose of suppressing mitochondrial ROS.

Following the finding that DNP effectively suppresses nuclear ROS (FIGS. 3A-3C), it is tested whether the use of DNP in combination with chemotherapeutic agents (e.g. doxorubicin or fulvestrant) provide better therapeutic effects against breast cancer xenografts than chemotherapies alone. Xenograft models are created from BT20, BT474 and MDA-MB231 cells injected into the mammary fat pad of female nude mice. To allow real time tracking of tumor growth and metastasis cells are stably transfected with luciferin. Detection is performed using whole body spectrometry/luminometry with an IVIS/LAGO instrument (see example in FIG. 2B). Treatment with DNP is initiated after tumors reach 200 mm³ in volume. The effect of DNP and combinations on tumor growth are assessed by measuring tumor dimensions every other day using a digital caliper. DNP is administered every other day at a low dose of 1 mg/Kg and a high dose 10 mg/Kg based on data generated from preclinical and ongoing clinical studies performed. In addition, combination treatments with doxorubicin or fulvestrant are tested, and compared with groups of mice treated with chemotherapy alone. In a non-limiting example, 10 mice per group are used to detect an effect size that is equal or higher than 0.5 of the mean standard deviation with α=0.05 and p<0.05 between groups.

To complement these studies, needle biopsies are used to retrieve tumor tissue from the mice. Tissues are fixated and analyzed by PWS and MSA to determine levels of chromatin folding heterogeneity before and after treatment. Hence, the first study of the effects of chromatin heterogeneity on the evolution of patient-derived tumor tissue is proposed. In addition, PWS is used as a monitoring tool to assess responsiveness to chemotherapy alone and in combination with DNP. According to data (e.g. FIG. 2A) it is expected that chemotherapy will increase ROS production promoting chromatin folding heterogeneity and the emergence of tumor cell phenotypes that ultimately resist chemotherapy and metastasize recapitulating most frequent clinical outcomes. It is also anticipated that the application of ROS scavengers targeting mitochondrial ROS will significantly restrict chromatin folding heterogenization by itself suppressing metastasis and tumor growth similarly to the effects of NLS-catalase shown in FIG. 2B. Hence, it is anticipated that in combination with chemotherapy, the use of DNP will result in tumor remission, a significant extension of life and an overall reduction in the rate of viable metastatic recurrence.

Data in FIGS. 3A-3C indicate that, in some embodiments, DNP suppresses nuclear ROS and might not work as a chromatin protective agent when tried in vivo. In some embodiments, the specific environment of the tumor (i.e. leaky vessels, poor perfusion, etc.) makes it challenging to deliver the drug to tumors in quantities that are sufficient to drive nuclear ROS levels down. In some embodiments, both NLS-Orp1roGFP2 knock in mice as well as cell lines were developed where different formulations and carriers can be tested in vivo for their ability to deliver DNP to tumors while also reporting on its effect to reduce nuclear ROS. In some embodiments, 2,4-dinitrophenyl morpholine-4-carboxylate, a precursor of DNP with different pharmacokinetics and pharmacodynamics that can be used in combination with or to replace DNP, is used as a therapeutic component.

### Example 2: Combination Therapy Using DNP Prodrugs

This Example demonstrates that the use of a DNP and/or a prodrug of DNP (e.g. a compound of any one of Formula I, Formula II, Formula III, Formula IV, Formula V, and Formula VI, including but not limited to 2,4-dinitrophenyl morpholine-4-carboxylate, 4-dinitrophenyl piperidine-1-carboxylate, 2,4-dinitrophenyl 4-methylpiperazine-1-carboxylate, 2,4-dinitrophenyl 4-(2-(piperidin-1-yl)ethoxy)benzoate) in combination with chemotherapeutic agents (e.g. doxorubicin or fulvestrant) provide better therapeutic effects against breast cancer xenografts than chemotherapies alone. Xenograft models are created from BT20, BT474 and MDA-MB231 cells injected into the mammary fat pad of female nude mice. To allow real time tracking of tumor growth and metastasis cells are stably transfected with luciferin. Detection is performed using whole body spectrometry/luminometry with an IVIS/LAGO instrument (see example in FIG. 2B). Treatment with a DNP prodrug is initiated after tumors reach 200 mm³ in volume. The effect of DNP prodrug and combinations on tumor growth are assessed by measuring tumor dimensions every other day using a digital caliper. DNP prodrug is administered every other day at a low dose of 1 mg/Kg and a high dose 10 mg/Kg based on data generated from preclinical and ongoing clinical studies performed. In addition, combination treatments with doxorubicin or fulvestrant are tested, and compared with groups of mice treated with chemotherapy alone. In a non-limiting example, 10 mice per group are used to detect an effect size that is equal or higher than 0.5 of the mean standard deviation with α=0.05 and p<0.05 between groups.

### REFERENCES

1. Takahashi, N. et al. Cancer Cells Co-opt the Neuronal Redox-Sensing Channel TRPA1 to Promote Oxidative-Stress Tolerance. Cancer Cell 33, 985-1003 e1007 (2018).
2. Li, F. et al. LKB1 Inactivation Elicits a Redox Imbalance to Modulate Non-small Cell Lung Cancer Plasticity and Therapeutic Response. Cancer Cell 27, 698-711 (2015).
3. Badgley, M. A. et al. Cysteine depletion induces pancreatic tumor ferroptosis in mice. Science 368, 85-89 (2020).
4. Suh, Y. A. et al. Cell transformation by the superoxide-generating oxidase Mox1. Nature 401, 79-82 (1999).
5. Pastushenko, I. et al. Fat1 deletion promotes hybrid EMT state, tumour stemness and metastasis. Nature 589, 448-455 (2021).
6. Rhim, A. D. et al. EMT and dissemination precede pancreatic tumor formation. Cell 148, 349-361 (2012).
7. Yu, M. et al. Circulating breast tumor cells exhibit dynamic changes in epithelial and mesenchymal composition. Science 339, 580-584 (2013).
8. Velarde, M. C., Flynn, J. M., Day, N. U., Melov, S. & Campisi, J. Mitochondrial oxidative stress caused by Sod2 deficiency promotes cellular senescence and aging phenotypes in the skin. Aging (Albany NY) 4, 3-12 (2012).
9. Sacktor, B. & Shimada, Y. Degenerative changes in the mitochondria of flight muscle from aging blowflies. J Cell Biol 52, 465-477 (1972).
10. Weindruch, R. H., Cheung, M. K., Verity, M. A. & Walford, R. L. Modification of mitochondrial respiration by aging and dietary restriction. Mech Ageing Dev 12, 375-392 (1980).
11. Jang, Y. C. & Van Remmen, H. The mitochondrial theory of aging: insight from transgenic and knockout mouse models. Exp Gerontol 44, 256-260 (2009).
12. Bekhet, O. H. & Eid, M. E. The interplay between reactive oxygen species and antioxidants in cancer progression and therapy: a narrative review. Transl Cancer Res 10, 4196-4206 (2021).
13. Bouyahya, A. et al. Preclinical and Clinical Antioxidant Effects of Natural Compounds against Oxidative Stress-Induced Epigenetic Instability in Tumor Cells. Antioxidants (Basel) 10 (2021).
14. Lambert, A. W. & Weinberg, R. A. Linking EMT programmes to normal and neoplastic epithelial stem cells. Nat Rev Cancer 21, 325-338 (2021).
15. Mabe, N. W. et al. Epigenetic silencing of tumor suppressor Par-4 promotes chemoresistance in recurrent breast cancer. J Clin Invest 128, 4413-4428 (2018).
16. Nachiyappan, A., Gupta, N. & Taneja, R. EHMT1/EHMT2 in EMT, cancer stemness and drug resistance: emerging evidence and mechanisms. FEBS J (2021).
17. Kabat, G. C. et al. Metabolic Obesity Phenotypes and Risk of Breast Cancer in Postmenopausal Women. Cancer Epidemiol Biomarkers Prev 26, 1730-1735 (2017).
18. Jones, D. P. & Go, Y. M. Redox compartmentalization and cellular stress. Diabetes Obes Metab 12 Suppl 2, 116-125 (2010).
19. Hu, J., Dong, L. & Outten, C. E. The redox environment in the mitochondrial intermembrane space is maintained separately from the cytosol and matrix. J Biol Chem 283, 29126-29134 (2008).
20. Kaludercic, N., Deshwal, S. & Di Lisa, F. Reactive oxygen species and redox compartmentalization. Front Physiol 5, 285 (2014).
21. Saeedi Saravi, S. S. et al. Differential endothelial signaling responses elicited by chemogenetic H₂O₂ synthesis. Redox Biol 36, 101605 (2020).
22. Truong, T. H. & Carroll, K. S. Redox regulation of protein kinases. Crit Rev Biochem Mol Biol 48, 332-356 (2013).
23. Cherkezyan, L. et al. Interferometric Spectroscopy of Scattered Light Can Quantify the Statistics of Subdiffractional Refractive-Index Fluctuations. Physical Review Letters 111 (2013).
24. Almassalha, L. M. et al. Label-free imaging of the native, living cellular nanoarchitecture using partial-wave spectroscopic microscopy. Proceedings of the National Academy of Sciences of the United States of America 113, E6372-E6381 (2016).
25. Cherkezyan, L. et al. Nanoscale changes in chromatin organization represent the initial steps of tumorigenesis: a transmission electron microscopy study. BMC Cancer 14 (2014).
26. Roy, H. K. et al. Optical Detection of Buccal Epithelial Nanoarchitectural Alterations in Patients Harboring Lung Cancer: Implications for Screening. Cancer Research 70, 7748-7754 (2010).
27. Backman, V. & Roy, H. K. Advances in Biophotonics Detection of Field Carcinogenesis for Colon Cancer Risk Stratification. Journal of Cancer 4, 251-261 (2013).
28. Konda, V. et al. Nanoscale Differences Assessed by Partial Wave Spectroscopic Microscopy in the Field of Esophageal Cancer and Barrett's Esophagus. Endoscopy 45, 983-988 (2013).
29. Damania, D. et al. Insights into the field carcinogenesis of ovarian cancer based on the nanocytology of endocervical and endometrial epithelial cells. International Journal of Cancer 133, 1143-1152 (2013).
30. Roy, H. K. et al. Nanocytological field carcinogenesis detection to mitigate overdiagnosis of prostate cancer: a proof of concept study. PloS one 10, e0115999-e0115999 (2015).
31. Damania, D. et al. Nanocytology of Rectal Colonocytes to Assess Risk of Colon Cancer Based on Field Cancerization. Cancer Research 72, 2720-2727 (2012).
32. Subramanian, H. et al. Procedures for risk-stratification of lung cancer using buccal nanocytology. Biomedical Optics Express 7, 3795-3810 (2016).
33. Subramanian, H. et al. Optical methodology for detecting histologically unapparent nanoscale consequences of genetic alterations in biological cells. PNAS 105, 20118-20123 (2008).
34. Stypula-Cyrus, Y. et al. HDAC Up-Regulation in Early Colon Field Carcinogenesis Is Involved in Cell Tumorigenicity through Regulation of Chromatin Structure. Plos One 8 (2013).
35. Cherkezyan, L., Subramanian, H. & Backman, V. What structural length scales can be detected by the spectral variance of a microscope image? Optics Letters 39, 4290-4293 (2014).
36. Cherkezyan, L., Zhang, D., Subramanian, H., Taflove, A. & Backman, V. Reconstruction of explicit structural properties at the nanoscale via spectroscopic microscopy. Journal of Biomedical Optics 21 (2016).
37. Cherkezyan, C. et al. Review of interferometric spectroscopy of scattered light for the quantification of subdiffractional structure of biomaterials. J. Biomed. Optics 22, 030901-030901-030919 (2017).
38. Agus, D. B. et al. A physical sciences network characterization of non-tumorigenic and metastatic cells. Scientific Reports 3 (2013).
39. Roy, H. K., Hensing, T. & Backman, V. Nanocytology for field carcinogenesis detection: novel paradigm for lung cancer risk stratification. Future Oncology 7, 1-3 (2011).
40. Roy, H. K. et al. Nano-Architectural Alterations in Mucus Layer Fecal Colonocytes in Field Carcinogenesis: Potential for Screening. Cancer Prevention Research 6, 1111-1119 (2013).
41. Subramanian, H. et al. Partial-wave microscopic spectroscopy detects subwavelength refractive index fluctuations: an application to cancer diagnosis. Optics Letters 34, 518-520 (2009).
42. Subramanian, H. et al. Nanoscale Cellular Changes in Field Carcinogenesis Detected by Partial Wave Spectroscopy. Cancer Research 69, 5357-5363 (2009).
43. Momi, N., Backman, V., Brendler, C. B. & Roy, H. K. Harnessing novel modalities: field carcinogenesis detection for personalizing prostate cancer management. Future oncology (London, England) 11, 2737-2741 (2015).
44. Almassalha, L. M. et al. Macrogenomic engineering by manipulating variations in chromatin folding. Nature Biomedical Engineering, under review.
45. Seebacher, N. A., Krchniakova, M., Stacy, A. E., Skoda, J. & Jansson, P. J. Tumour Microenvironment Stress Promotes the Development of Drug Resistance. Antioxidants (Basel) 10 (2021).
46. Shen, C. J. et al. Oleic acid-induced NOX4 is dependent on ANGPTL4 expression to promote human colorectal cancer metastasis. Theranostics 10, 7083-7099 (2020).
47. van 't Erve T J, Kadiiska M B, London S J, Mason R P. Classifying oxidative stress by F2-isoprostane levels across human diseases: A meta-analysis. Redox Biol. 2017; 12:582-99. Epub 2017 Apr. 10. doi: 10.1016/j.redox.2017.03.024. PubMed PMID: 28391180; PMCID: PMC5384299.

We claim:

1. A method of treating cancer comprising:
administering to a patient in need thereof a therapeutic amount of dinitrophenol (DNP) or a pharmaceutically acceptable salt thereof,
wherein the therapeutic amount of DNP is in the range of about 0.01 mg/kg of body weight to about 20 mg/kg of body weight.

2. The method of claim 1, wherein the therapeutic amount of DNP is in the range of about 0.001 mg/kg of body weight to about 15 mg/kg of body weight, about 0.01 mg/kg of body weight to about 15 mg/kg of body weight, 0.1 mg/kg of body weight to about 15 mg/kg of body weight, about 0.001 mg/kg of body weight to about 0.1 mg/kg of body weight, about 0.001 mg/kg of body weight to about 0.01 mg/kg of body weight, about 0.01 mg/kg of body weight to about 0.1 mg/kg of body weight, about 1 mg/kg of body weight to about 10 mg/kg of body weight, about 3 mg/kg of body weight to about 8 mg/kg of body weight, or about 4 mg/kg of body weight to about 6 mg/kg of body weight.

3. The method of claim 1 or 2, wherein the therapeutic amount of DNP is about 0.001 mg/kg, about 0.005 mg/kg, about 0.01 mg/kg, about 0.05 mg/kg, about 0.1 mg/kg, about 0.5 mg/kg, 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, or about 10 mg/kg of body weight.

4. A method of treating cancer comprising:
administering to a patient in need thereof a therapeutic amount of a prodrug of dinitrophenol (DNP) or a pharmaceutically acceptable salt thereof,
wherein the therapeutic amount of the prodrug of DNP is in the range of about 0.01 mg/kg of body weight to about 200 mg/kg of body weight.

5. The method of claim 4, wherein the therapeutic amount of the prodrug of DNP is in the range of about 0.001 mg/kg of body weight to about 15 mg/kg of body weight, about 0.01 mg/kg of body weight to about 15 mg/kg of body weight, about 0.001 mg/kg of body weight to about 100 mg/kg of body weight, about 0.01 mg/kg of body weight to about 100 mg/kg of body weight, about 0.1 mg/kg of body weight to about 15 mg/kg of body weight, about 0.001 mg/kg of body weight to about 0.1 mg/kg of body weight, about 0.001 mg/kg of body weight to about 0.01 mg/kg of body weight, about 0.01 mg/kg of body weight to about 0.1 mg/kg of body weight, about 0.1 mg/kg of body weight to about 100 mg/kg of body weight, about 1 mg/kg of body weight to about 100 mg/kg of body weight, about 1 mg/kg of body weight to about 20 mg/kg of body weight, about 5 mg/kg of body weight to about 10 mg/kg of body weight, about 7 mg/kg of body weight to about 9 mg/kg of body weight, about 10 mg/kg of body weight to about 20 mg/kg of body weight, about 15 mg/kg of body weight to about 17 mg/kg of body weight, about 50 mg/kg of body weight to about 100 mg/kg of body weight, about 60 mg/kg of body weight to about 90 mg/kg of body weight, about 75 mg/kg of body weight to about 85 mg/kg of body weight, or about 79 mg/kg of body weight to about 81 mg/kg of body weight.

6. The method of claim 4 or 5, wherein the therapeutic amount of the prodrug of DNP is about 0.001 mg/kg of body weight, about 0.005 mg/kg of body weight, about 0.01 mg/kg of body weight, about 0.05 mg/kg of body weight, about 0.1 mg/kg of body weight, about 0.5 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, or about 20 mg/kg of body weight.

7. The method of any one of claims 4-6, wherein the therapeutic amount of the prodrug of DNP is about 71 mg/kg of body weight, about 72 mg/kg of body weight, about 73 mg/kg of body weight, about 74 mg/kg of body weight, about 75 mg/kg of body weight, about 76 mg/kg of body weight, about 77 mg/kg of body weight, about 78 mg/kg of body weight, about 79 mg/kg of body weight, 80 mg/kg of body weight, about 81 mg/kg of body weight, about 82 mg/kg of body weight, about 83 mg/kg of body weight, about 84 mg/kg of body weight, about 85 mg/kg of body weight, about 86 mg/kg of body weight, about 87 mg/kg of body weight, about 88 mg/kg of body weight, about 89 mg/kg of body weight, or about 90 mg/kg of body weight.

8. The method of any one of claims 4-7, wherein the DNP is selected from 2,3-DNP, 2,4-DNP, 2,5-DNP, 2,6-DNP, 3,4-DNP, or 3,5-DNP.

9. The method of any one of claims 4-8, wherein the DNP is 2,4-DNP.

10. The method of any one of claims 4-7, wherein the prodrug of DNP is selected from:
an amino acid (AA) ester of 2,3-DNP, an amino acid (AA) ester of 2,4-DNP, an amino acid (AA) ester of 2,5-DNP, an amino acid (AA) ester of 2,6-DNP, an amino acid (AA) ester of 3,4-DNP, or an amino acid (AA) ester of 3,5-DNP (Scheme 1, Formulas I-1 to I-10; Scheme 2, Formulas II-1 to II-10; Scheme 3, Formulas III-1 to III-10; Scheme 4, Formulas IV-1 to IV-10; Scheme 5, Formulas V-1 to V-10; and Scheme 6, Formulas VI-1 to VI-10);
AA esters incorporating a methylene dioxide (a formaldehyde equivalent) spacer (Scheme 1, Formulas I-11 to I-13; Scheme 2, Formulas II-11 to II-13; Scheme 3, Formulas III-11 to III-13; Scheme 4, Formulas IV-11 to IV-13; Scheme 5, Formulas V-11 to V-13; and Scheme 6, Formulas VI-11 to VI-13);
amino carbamate 2,3-DNP prodrugs, amino carbamate 2,4-DNP prodrugs, amino carbamate 2,5-DNP prodrugs, amino carbamate 2,6-DNP prodrugs, amino carbamate 3,4-DNP prodrugs, or amino carbamate 3,5-DNP prodrugs (Scheme 1, Formulas I-14 to I-17; Scheme 2, Formulas II-14 to II-17; Scheme 3, Formulas III-14 to III-17; Scheme 4, Formulas IV-14 to IV-17; Scheme 5, Formulas V-14 to V-17; and Scheme 6, Formulas VI-14 to VI-17);
amino carbonate 2,3-DNP prodrugs, amino carbonate 2,4-DNP prodrugs, amino carbonate 2,5-DNP prodrugs, amino carbonate 2,6-DNP prodrugs, amino carbonate 3,4-DNP prodrugs, or amino carbonate 3,5-DNP prodrugs (Scheme 1, Formulas I-18 and I-19; Scheme 2, Formulas II-18 and II-19; Scheme 3, Formulas III-18 and III-19; Scheme 4, Formulas IV-18 and IV-19; Scheme 5, Formulas V-18 and V-19; and Scheme 6, Formulas VI-18 and VI-19);
phosphate analogs I-20, I-21, II-20, II-21, III-20, III-21, IV-20, IV-21, V-20, V-21, VI-20, and VI-21 (Schemes 1-6);
1,3 diketo analogs I-22 to I-32; II-22 to II-32; III-22 to III-32; IV-22 to IV-32; V-22 to V-32; and VI-22 to VI-32 (Schemes 1-6);
carbonate and carbamate analogs I-33 to I-39; II-33 to II-39; III-33 to III-39; IV-33 to IV-39; V-33 to V-39; and VI-33 to VI-39 (Schemes 1-6); and
benzoate analogs I-40, II-40, III-40, IV-40, V-40, and VI-40 (Schemes 1-6);
and combinations thereof.

11. The method of any one of claims 4-7, wherein the prodrug of DNP is selected from:

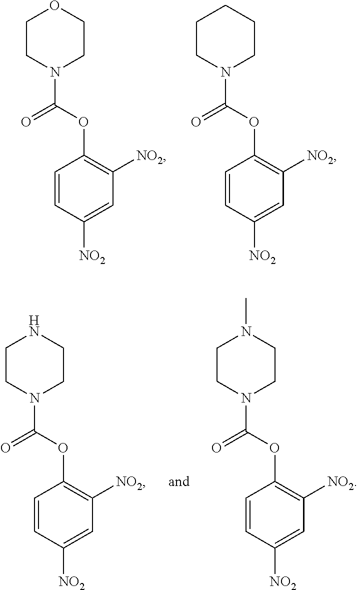

12. The method of any one of claims 1-11, wherein the DNP and/or the prodrug of DNP is administered orally.

13. The method of any one of claims 1-11, wherein the DNP and/or the prodrug of DNP is administered intravenously, subcutaneously, or transdermally.

14. The method of any one of claims 1-13, wherein the DNP and/or the prodrug of DNP is administered at a frequency of once a day, twice a day, three times a day, four times a day, once every two days, once every three days, once every four days, once every five days, once every six days, once a week, once every two weeks, once every three weeks, or once a month.

15. The method of any one of claims 1-14, wherein the DNP and/or the prodrug of DNP is administered at a frequency of once a day.

16. The method of claim 15, wherein the DNP and/or the prodrug of DNP is administered at a frequency of once a day for one day, once a day for two consecutive days, once a day for three consecutive days, once a day for four consecutive days, once a day for five consecutive days, once a day for six consecutive days, once a day for 7 consecutive days, or once a day for two consecutive weeks.

17. The method of any one of claims 1-16, wherein the cancer is selected from breast cancer (including pre- and post-menopausal breast cancer), blood cancer, cancer of the lymphatic system, bone cancer, brain cancer, cervical cancer, childhood cancer, colorectal cancer, head and/or neck cancer, stomach cancer, esophageal cancer, kidney cancer, liver cancer, lung cancer, metastatic cancer, ovarian cancer, pancreatic cancer, prostate cancer, sarcoma, skin cancer, testicular cancer, thyroid cancer, uterine cancer, lymphoma, colon cancer, melanoma, malignant melanoma, primary brain carcinoma, head-neck cancer, glioma, glioblastoma, bladder cancer, non-small cell lung cancer, breast carcinoma, ovarian carcinoma, small-cell lung carcinoma, Wilms' tumor, cervical carcinoma, bladder carcinoma, pancreatic carcinoma, colon carcinoma, prostatic carcinoma, genitourinary carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, myeloid leukemia, acute myeloid leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma.

18. The method of any one of claims 1-16, wherein the cancer is breast cancer (including pre- and post-menopausal breast cancer, and metastatic breast cancer).

19. The method of any one of claims 1-16, wherein the cancer is selected from lung cancer, pancreatic cancer, and prostate cancer.

20. The method of any one of claims 11-19, wherein the DNP and/or the prodrug of DNP is administered in combination with an additional therapeutic agent.

21. The method of claim 20, wherein the additional therapeutic agent comprises chemotherapy.

22. The method of claim 20 or 21, wherein the additional therapeutic agent is selected from abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dalteparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, geldanamycin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, paclitaxel, pegaspargase, pegfilgrastim, peginterferon alpha-2b, pemetrexed, pentostatin, pipobroman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, and analogues thereof.

23. The method of claim 20 or 21, wherein the additional therapeutic agent is doxorubicin or an analogue thereof.

24. The method of claim 20 or 21, wherein the additional therapeutic agent is fulvestrant or an analogue thereof.

25. The method of claim 20 or 21, wherein the additional therapeutic agent is paclitaxel or an analogue thereof.

26. The method of claim 20 or 21, wherein the additional therapeutic agent is docetaxel or an analogue thereof.

27. The method of any one of claims 1-20, wherein the DNP and/or the prodrug of DNP is administered without chemotherapy.

28. The method of claim 20, wherein the additional therapeutic agent comprises radiation therapy.
